SEQUENCE LISTING (10 sequences)

SEQ ID NO: 1 - DNA, 1614 bp, Artificial sequence
  pSCHIZ Sec1-Invertase
ggatccatgaagttcgcgacctcggtcgcaattttgcttgtggccaacatagccaccgccctcgcgtcgatgaccaacgagacctcggaccgccctctcgtgcactttacccccaacaagggttggatgaacgatcccaacggcctctggtacgacgagaaggatgctaagtggcacctttactttcagtacaaccctaacgacaccgtctggggcaccccgctcttctggggccacgccacctccgacgacctcaccaactgggaggaccagcccattgctatcgcccccaagcgcaacgactcgggagctttttccggttccatggttgtggactacaacaacacctccggtttttttaacgacaccattgacccccgccagcgctgcgtcgccatctggacctacaacacgcccgagagcgaggagcagtacatcagctacagccttgatggaggctacacctttaccgagtaccagaagaaccctgtcctcgccgccaactccacccagttccgcgaccctaaggttttttggtacgagccttcccagaagtggattatgaccgccgctaagtcgcaggattacaagatcgagatctacagcagcgacgacctcaagtcctggaagcttgagtccgcctttgccaacgagggttttctcggataccagtacgagtgccccggtctcatcgaggtccccaccgagcaggacccgtccaagtcctactgggtcatgtttatttccatcaaccctggcgcccctgccggcggcagcttcaaccagtacttcgtcggctcctttaacggcacgcattttgaggccttcgacaaccagtcccgcgtcgtcgacttcggcaaggactactacgccctccagaccttctttaacaccgaccccacctacggcagcgccctcggtattgcttgggcctccaactgggagtactccgctttcgtccccactaacccctggcgcagctcgatgtccctcgtccgcaagttttcgcttaacaccgagtaccaggccaaccccgagaccgagcttattaacctgaaggccgagcctattctcaacatctccaacgctggcccctggtcccgctttgctactaacactaccctcaccaaggccaactcctacaacgtcgatctctccaactccaccggtactcttgagtttgagctcgtctacgccgtcaacaccacccagaccatctccaagtccgtcttcgccgacctctccctctggttcaagggccttgaggaccccgaggagtacctgcgcatgggttttgaggtctccgcctcctccttcttcctcgatcgcggtaactccaaggttaagtttgtcaaggagaacccctactttactaaccgtatgagcgtcaacaaccagccctttaagtccgagaacgatcttagctactacaaggtttacggcctcctcgaccagaacattctcgagctctactttaacgacggagatgtcgtcagcaccaacacctactttatgaccactggaaacgccctcggcagcgtgaacatgaccaccggagtcgacaacctcttttacattgacaagtttcaggttcgcgaggttaagtaacatatg

SEQ ID NO: 2 - DNA, 11495 bp, Artificial sequence
  pCL0076
ctcttatctgcctcgcgccgttgaccgccgcttgactcttggcgcttgccgctcgcatcctgcctcgctcgcgcaggcgggcgggcgagtgggtgggtccgcagccttccgcgctcgcccgctagctcgctcgcgccgtgctgcagccagcagggcagcaccgcacggcaggcaggtcccggcgcggatcgatcgatccatcgatccatcgatccatcgatcgtgcggtcaaaaagaaaggaagaagaaaggaaaaagaaaggcgtgcgcacccgagtgcgcgctgagcgcccgctcgcggtcccgcggagcctccgcgttagtccccgccccgcgccgcgcagtcccccgggaggcatcgcgcacctctcgccgccccctcgcgcctcgccgattccccgcctccccttttccgcttcttcgccgcctccgctcgcggccgcgtcgcccgcgccccgctccctatctgctccccaggggggcactccgcaccttttgcgcccgctgccgccgccgcggccgccccgccgccctggtttcccccgcgagcgcggccgcgtcgccgcgcaaagactcgccgcgtgccgccccgagcaacgggtggcggcggcgcggcggcgggcggggcgcggcggcgcgtaggcggggctaggcgccggctaggcgaaacgccgcccccgggcgccgccgccgcccgctccagagcagtcgccgcgccagaccgccaacgcagagaccgagaccgaggtacgtcgcgcccgagcacgccgcgacgcgcggcagggacgaggagcacgacgccgcgccgcgccgcgcggggggggggagggagaggcaggacgcgggagcgagcgtgcatgtttccgcgcgagacgacgccgcgcgcgctggagaggagataaggcgcttggatcgcgagagggccagccaggctggaggcgaaaatgggtggagaggatagtatcttgcgtgcttggacgaggagactgacgaggaggacggatacgtcgatgatgatgtgcacagagaagaagcagttcgaaagcgactactagcaagcaagggatccatgaagttcgcgacctcggtcgcaattttgcttgtggccaacatagccaccgccctcgcgtcgatgaccaacgagacctcggaccgccctctcgtgcactttacccccaacaagggttggatgaacgatcccaacggcctctggtacgacgagaaggatgctaagtggcacctttactttcagtacaaccctaacgacaccgtctggggcaccccgctcttctggggccacgccacctccgacgacctcaccaactgggaggaccagcccattgctatcgcccccaagcgcaacgactcgggagctttttccggttccatggttgtggactacaacaacacctccggtttttttaacgacaccattgacccccgccagcgctgcgtcgccatctggacctacaacacgcccgagagcgaggagcagtacatcagctacagccttgatggaggctacacctttaccgagtaccagaagaaccctgtcctcgccgccaactccacccagttccgcgaccctaaggttttttggtacgagccttcccagaagtggattatgaccgccgctaagtcgcaggattacaagatcgagatctacagcagcgacgacctcaagtcctggaagcttgagtccgcctttgccaacgagggttttctcggataccagtacgagtgccccggtctcatcgaggtccccaccgagcaggacccgtccaagtcctactgggtcatgtttatttccatcaaccctggcgcccctgccggcggcagcttcaaccagtacttcgtcggctcctttaacggcacgcattttgaggccttcgacaaccagtcccgcgtcgtcgacttcggcaaggactactacgccctccagaccttctttaacaccgaccccacctacggcagcgccctcggtattgcttgggcctccaactgggagtactccgctttcgtccccactaacccctggcgcagctcgatgtccctcgtccgcaagttttcgcttaacaccgagtaccaggccaaccccgagaccgagcttattaacctgaaggccgagcctattctcaacatctccaacgctggcccctggtcccgctttgctactaacactaccctcaccaaggccaactcctacaacgtcgatctctccaactccaccggtactcttgagtttgagctcgtctacgccgtcaacaccacccagaccatctccaagtccgtcttcgccgacctctccctctggttcaagggccttgaggaccccgaggagtacctgcgcatgggttttgaggtctccgcctcctccttcttcctcgatcgcggtaactccaaggttaagtttgtcaaggagaacccctactttactaaccgtatgagcgtcaacaaccagccctttaagtccgagaacgatcttagctactacaaggtttacggcctcctcgaccagaacattctcgagctctactttaacgacggagatgtcgtcagcaccaacacctactttatgaccactggaaacgccctcggcagcgtgaacatgaccaccggagtcgacaacctcttttacattgacaagtttcaggttcgcgaggttaagtaacatatgttatgagagatccgaaagtgaaccttgtcctaacccgacagcgaatggcgggagggggcgggctaaaagatcgtattacatagtatttttcccctactctttgtgtttgtcttttttttttttttgaacgcattcaagccacttgtctgggtttacttgtttgtttgcttgcttgcttgcttgcttgcctgcttcttggtcagacggcccaaaaaagggaaaaaattcattcatggcacagataagaaaaagaaaaagtttgtcgaccaccgtcatcagaaagcaagagaagagaaacactcgcgctcacattctcgctcgcgtaagaatcttagccacgcatacgaagtaatttgtccatctggcgaatctttacatgagcgttttcaagctggagcgtgagatcatacctttcttgatcgtaatgttccaaccttgcataggcctcgttgcgatccgctagcaatgcgtcgtactcccgttgcaactgcgccatcgcctcattgtgacgtgagttcagattcttctcgagaccttcgagcgctgctaatttcgcctgacgctccttcttttgtgcttccatgacacgccgcttcaccgtgcgttccacttcttcctcagacatgcccttggctgcctcgacctgctcggtaagcttcgtcgtaatctcctcgatctcggaattcttcttgccctccatccactcggcaccatacttggcagcctgttcaacacgctcattgaaaaacttttcattctcttccagctccgcaacccgcgctcgaagctcattcacttccgccaccacggcttcggcatcgagcgccgaatcagtcgccgaactttccgaaagatacaccacggcccctccgctgctgctgcgcagcgtcatcatcagtcgcgtgttatcttcgcgcagattctccacctgctccgtaagcagcttcacggtggcctcttgattctgagggctcacgtcgtggattagcgcttgcagctcttgcagctccgtcagcttggaagagctcgtaatcatggctttgcacttgtccagacgtcgcagagcgttcgagagccgcttcgcgttatctgccatggacgcttctgcgctcgcggcctccctgacgacagtctcttgcagtttcactagatcatgtccaatcagcttgcggtgcagctctccaatcacgttctgcatcttgtttgtgtgtccgggccgcgcctcgtcttgcgatttgcgaatttcctcctcgagctcgcgttcgagctccagggcgcctttaagtagctcgaagtcagccgccgttagccccagctccgtcgccgcgttcagacagtcggttagcttgattcgattccgcttttccatggcaagtttaagatcctggcccagctgcacctcctgcgccttgcgcatcatgcgcggttccgcctggcgcaaaagcttcgagtcgtatcctgcctgccatgccagcgcaatggcacgcacgagcgacttgagttgccaactattcatcgccgagatgagcagcattttgatctgcatgaacacctcgtcagagtcgtcatcctctgcctcctccagctctgcgggcgagcgacgctctccttgcagatgaagcgagggccgcaggcctccgaagagcacctcttgcgcgagatcctcctccgtcgtcgccctccgcaggattgcggtcgtgtccgccatcttgccgccacagcagcttttgctcgctctgcaccttcaatttctggtgccgctggtgccgctggtgccgcttgtgctggtgctggtgctggtgctggtgctggtgccttgtgctggtgctgccacagacaccgccgctcctgctgctgctcttccggccccctcgccgccgccgcgagcccccgccgcgcgccgtgcctgggctctccgcgctctccgcgggctcctcggcctcggcctcgccgtccgcgacgacgtctgcgcggccgatggtgcggatctgctctagagggcccttcgaaggtaagcctatccctaaccctctcctcggtctcgattctacgcgtaccggtcatcatcaccatcaccattgagtttaaacgggccccagcacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatacatggtcgacctgcaggaacctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccaagctttgcctcaacgcaactaggcccaggcctactttcactgtgtcttgtcttgcctttcacaccgaccgagtgtgcacaaccgtgttttgcacaaagcgcaagatgctcactcgactgtgaagcaaaggttgcgcgcaagcgactgcgactgcgaggatgaggatgactggcagcctgttcaaaaactgaaaatccgcgatgggtcagctgccattcgcgcatgacgcctgcgagagacaagttaactcgtgtcactggcatgtcctagcatctttacgcgagcaaaattcaatcgctttattttttcagtttcgtaaccttctcgcaaccgcgaatcgccgtttcagcctgactaatctgcagctgcgtggcactgtcagtcagtcagtcagtcgtgcgcgctgttccagcaccgaggtcgcgcgtcgccgcgcctggaccgctgctgctactgctagtggcacggcaggtaggagcttgttgccggaacaccagcagccgccagtcgacgccagccaggggaaagtccggcgtcgaagggagaggaaggcggcgtgtgcaaactaacgttgaccactggcgcccgccgacacgagcaggaagcaggcagctgcagagcgcagcgcgcaagtgcagaatgcgcgaaagatccacttgcgcgcggcgggcgcgcacttgcgggcgcggcgcggaacagtgcggaaaggagcggtgcagacggcgcgcagtgacagtgggcgcaaagccgcgcagtaagcagcggcggggaacggtatacgcagtgccgcgggccgccgcacacagaagtatacgcgggccgaagtggggcgtcgcgcgcgggaagtgcggaatggcgggcaaggaaaggaggagacggaaagagggcgggaaagagagagagagagagtgaaaaaagaaagaaagaaagaaagaaagaaagaaagctcggagccacgccgcggggagagagagaaatgaaagcacggcacggcaaagcaaagcaaagcagacccagccagacccagccgagggaggagcgcgcgcaggacccgcgcggcgagcgagcgagcacggcgcgcgagcgagcgagcgagcgagcgcgcgagcgagcaaggcttgctgcgagcgatcgagcgagcgagcgggaaggatgagcgcgacccgcgcggcgacgaggacagcggcggcgctgtcctcggcgctgacgacgcctgtaaagcagcagcagcagcagcagctgcgcgtaggcgcggcgtcggcacggctggcggccgcggcgttctcgtccggcacgggcggagacgcggccaagaaggcggccgcggcgagggcgttctccacgggacgcggccccaacgcgacacgcgagaagagctcgctggccacggtccaggcggcgacggacgatgcgcgcttcgtcggcctgaccggcgcccaaatctttcatgagctcatgcgcgagcaccaggtggacaccatctttggctaccctggcggcgccattctgcccgtttttgatgccatttttgagagtgacgcgcttcaagttcattctcgctcgccacgagcagggcgccggccacatggccgagggctacgcgcgcgccacgggcaagcccggcgttgtcctcgtcacctcgggccctggagccaccaacaccatcaccccgatcatggatgcttacatggacggtacgccgctgctcgtgttcaccggccaggtgcagacctctgctgtcggcacggacgctttccaggagtgtgacattgttggcatcagccgcgcgtgcaccaagtggaacgtcatggtcaaggacgtgaaggagctcccgcgccgcatcaatgaggcctttgagattgccatgagcggccgcccgggtcccgtgctcgtcgatcttcctaaggatgtgaccgccgttgagctcaaggaaatgcccgacagctccccccaggttgctgtgcgccagaagcaaaaggtcgagcttttccacaaggagcgcattggcgctcctggcacggccgacttcaagctcattgccgagatgatcaaccgtgcggagcgacccgtcatctatgctggccagggtgtcatgcagagcccgttgaatggcccggctgtgctcaaggagttcgcggagaaggccaacattcccgtgaccaccaccatgcagggtctcggcggctttgacgagcgtagtcccctctccctcaagatgctcggcatgcacggctctgcctacgccaactactcgatgcagaacgccgatcttatcctggcgctcggtgcccgctttgatgatcgtgtgacgggccgcgttgacgcctttgctccggaggctcgccgtgccgagcgcgagggccgcggtggcatcgttcactttgagatttcccccaagaacctccacaaggtcgtccagcccaccgtcgcggtcctcggcgacgtggtcgagaacctcgccaacgtcacgccccacgtgcagcgccaggagcgcgagccgtggtttgcgcagatcgccgattggaaggagaagcacccttttctgctcgagtctgttgattcggacgacaaggttctcaagccgcagcaggtcctcacggagcttaacaagcagattctcgagattcaggagaaggacgccgaccaggaggtctacatcaccacgggcgtcggaagccaccagatgcaggcagcgcagttccttacctggaccaagccgcgccagtggatctcctcgggtggcgccggcactatgggctacggccttccctcggccattggcgccaagattgccaagcccgatgctattgttattgacatcgatggtgatgcttcttattcgatgaccggtatggaattgatcacagcagccgaattcaaggttggcgtgaagattcttcttttgcagaacaactttcagggcatggtcaagaacgttcaggatctcttttacgacaagcgctactcgggccaccgccatgttcaacccgcgcttcgacaaggtcgccgatgcgatgcgtgccaagggtctctactgcgcgaaacagtcggagctcaaggacaagatcaaggagtttctcgagtacgatgagggtcccgtcctcctcgaggttttcgtggacaaggacacgctcgtcttgcccatggtccccgctggctttccgctccacgagatggtcctcgagcctcctaagcccaaggacgcctaagttcttttttccatggcgggcgagcgagcgagcgcgcgagcgcgcaagtgcgcaagcgccttgccttgctttgcttcgcttcgctttgctttgcttcacacaacctaagtatgaattcaagttttcttgcttgtcggcgatgcctgcctgccaaccagccagccatccggccggccgtccttgacgccttcgcttccggcgcggccatcgattcaattcacccatccgatacgttccgccccctcacgtccgtctgcgcacgacccctgcacgaccacgccaaggccaacgcgccgctcagctcagcttgtcgacgagtcgcacgtcacatatctcagatgcatttggactgtgagtgttattatgccactagcacgcaacgatcttcggggtcctcgctcattgcatccgttcgggccctgcaggcgtggacgcgagtcgccgccgagacgctgcagcaggccgctccgacgcgagggctcgagctcgccgcgcccgcgcgatgtctgcctggcgccgactgatctctggagcgcaaggaagacacggcgacgcgaggaggaccgaagagagacgctggggtatgcaggatatacccggggcgggacattcgttccgcatacactcccccattcgagcttgctcgtccttggcagagccgagcgcgaacggttccgaacgcggcaaggattttggctctggtgggtggactccgatcgaggcgcaggttctccgcaggttctcgcaggccggcagtggtcgttagaaatagggagtgccggagtcttgacgcgccttagctcactctccgcccacgcgcgcatcgccgccatgccgccgtcccgtctgtcgctgcgctggccgcgaccggctgcgccagagtacgacagtgggacagagctcgaggcgacgcgaatcgctcgggttgtaagggtttcaagggtcgggcgtcgtcgcgtgccaaagtgaaaatagtaggggggggggggggtac

SEQ ID NO: 3 - protein, 20 aa, Schizochytrium
MKFATSVAILLVANIATALA

SEQ ID NO: 4 - DNA, 1785 bp, Artificial
  Codon optimized nucleic acid sequence
atgaagttcgcgacctcggtcgcaattttgcttgtggccaacatagccaccgccctcgcggcctccccctcgatgcagacccgtgcctccgtcgtcattgattacaacgtcgctcctcctaacctctccaccctcccgaacggcagcctctttgagacctggcgtcctcgcgcccacgttcttccccctaacggtcagattggcgatccctgcctccactacaccgatccctcgactggcctctttcacgtcggctttctccacgatggctccggcatttcctccgccactactgacgacctcgctacctacaaggatctcaaccagggcaaccaggtcatcgtccccggcggtatcaacgaccctgtcgctgttttcgacggctccgtcattccttccggcattaacggcctccctaccctcctctacacctccgtcagctacctccccattcactggtccatcccctacacccgcggttccgagacgcagagcctggctgtctccagcgatggtggctccaactttactaagctcgaccagggccccgttattcctggccccccctttgcctacaacgtcaccgccttccgcgacccctacgtctttcagaaccccaccctcgactccctcctccactccaagaacaacacctggtacaccgtcatttcgggtggcctccacggcaagggccccgcccagtttctttaccgtcagtacgaccccgactttcagtactgggagttcctcggccagtggtggcacgagcctaccaactccacctggggcaacggcacctgggccggccgctgggccttcaacttcgagaccggcaacgtcttttcgcttgacgagtacggctacaacccccacggccagatcttctccaccattggcaccgagggctccgaccagcccgttgtcccccagctcacctccatccacgatatgctttgggtctccggtaacgtttcgcgcaacggatcggtttccttcactcccaacatggccggcttcctcgactggggtttctcgtcctacgccgccgcgggtaaggttcttccttccacgtcgctcccctccaccaagtccggtgcccccgatcgcttcatttcgtacgtttggctctccggcgacctctttgagcaggctgagggctttcctaccaaccagcagaactggaccggcaccctcctcctcccccgtgagctccgcgtcctttacatccccaacgtggttgataacgcccttgcgcgcgagtccggcgcttcctggcaggtcgtctcctccgatagctcggccggtactgtggagctccagaccctcggcatttccatcgcccgcgagaccaaggccgccctcctgtccggcacctcgttcactgagtccgaccgcactcttaactcctccggcgtcgttccctttaagcgttccccctccgagaagtttttcgtcctctccgcccagctctccttccccgcctccgcccgcggctcgggcctcaagtccggcttccagattctttcctccgagctcgagtccaccacggtctactaccagtttagcaacgagtccatcatcgtcgaccgcagcaacaccagcgccgccgcccgtactaccgacggtatcgactcctccgccgaggccggcaagctccgcctctttgacgtcctcaacggcggcgagcaggctattgagaccctcgaccttaccctcgtcgttgataactccgtgctcgagatttacgccaacggtcgtttcgcgctttccacctgggttcgctaa

SEQ ID NO: 5 - DNA, 9291 bp, Artificial Sequence
  pCL0120
ctcttatctgcctcgcgccgttgaccgccgcttgactcttggcgcttgccgctcgcatcctgcctcgctcgcgcaggcgggcgggcgagtgggtgggtccgcagccttccgcgctcgcccgctagctcgctcgcgccgtgctgcagccagcagggcagcaccgcacggcaggcaggtcccggcgcggatcgatcgatccatcgatccatcgatccatcgatcgtgcggtcaaaaagaaaggaagaagaaaggaaaaagaaaggcgtgcgcacccgagtgcgcgctgagcgcccgctcgcggtcccgcggagcctccgcgttagtccccgccccgcgccgcgcagtcccccgggaggcatcgcgcacctctcgccgccccctcgcgcctcgccgattccccgcctccccttttccgcttcttcgccgcctccgctcgcggccgcgtcgcccgcgccccgctccctatctgctccccaggggggcactccgcaccttttgcgcccgctgccgccgccgcggccgccccgccgccctggtttcccccgcgagcgcggccgcgtcgccgcgcaaagactcgccgcgtgccgccccgagcaacgggtggcggcggcgcggcggcgggcggggcgcggcggcgcgtaggcggggctaggcgccggctaggcgaaacgccgcccccgggcgccgccgccgcccgctccagagcagtcgccgcgccagaccgccaacgcagagaccgagaccgaggtacgtcgcgcccgagcacgccgcgacgcgcggcagggacgaggagcacgacgccgcgccgcgccgcgcggggggggggagggagaggcaggacgcgggagcgagcgtgcatgtttccgcgcgagacgacgccgcgcgcgctggagaggagataaggcgcttggatcgcgagagggccagccaggctggaggcgaaaatgggtggagaggatagtatcttgcgtgcttggacgaggagactgacgaggaggacggatacgtcgatgatgatgtgcacagagaagaagcagttcgaaagcgactactagcaagcaagggatccatgaagttcgcgacctcggtcgcaattttgcttgtggccaacatagccaccgccctcgcgcagagcgatggctgcacccccaccgaccagacgatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctgggacacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagcaccaccatcaccaccactaacatatgagttatgagatccgaaagtgaaccttgtcctaacccgacagcgaatggcgggagggggcgggctaaaagatcgtattacatagtatttttcccctactctttgtgtttgtcttttttttttttttgaacgcattcaagccacttgtctgggtttacttgtttgtttgcttgcttgcttgcttgcttgcctgcttcttggtcagacggcccaaaaaagggaaaaaattcattcatggcacagataagaaaaagaaaaagtttgtcgaccaccgtcatcagaaagcaagagaagagaaacactcgcgctcacattctcgctcgcgtaagaatcttagccacgcatacgaagtaatttgtccatctggcgaatctttacatgagcgttttcaagctggagcgtgagatcatacctttcttgatcgtaatgttccaaccttgcataggcctcgttgcgatccgctagcaatgcgtcgtactcccgttgcaactgcgccatcgcctcattgtgacgtgagttcagattcttctcgagaccttcgagcgctgctaatttcgcctgacgctccttcttttgtgcttccatgacacgccgcttcaccgtgcgttccacttcttcctcagacatgcccttggctgcctcgacctgctcggtaaaacgggccccagcacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatacatggtcgacctgcaggaacctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccaagctttgcctcaacgcaactaggcccaggcctactttcactgtgtcttgtcttgcctttcacaccgaccgagtgtgcacaaccgtgttttgcacaaagcgcaagatgctcactcgactgtgaagcaaaggttgcgcgcaagcgactgcgactgcgaggatgaggatgactggcagcctgttcaaaaactgaaaatccgcgatgggtcagctgccattcgcgcatgacgcctgcgagagacaagttaactcgtgtcactggcatgtcctagcatctttacgcgagcaaaattcaatcgctttattttttcagtttcgtaaccttctcgcaaccgcgaatcgccgtttcagcctgactaatctgcagctgcgtggcactgtcagtcagtcagtcagtcgtgcgcgctgttccagcaccgaggtcgcgcgtcgccgcgcctggaccgctgctgctactgctagtggcacggcaggtaggagcttgttgccggaacaccagcagccgccagtcgacgccagccaggggaaagtccggcgtcgaagggagaggaaggcggcgtgtgcaaactaacgttgaccactggcgcccgccgacacgagcaggaagcaggcagctgcagagcgcagcgcgcaagtgcagaatgcgcgaaagatccacttgcgcgcggcgggcgcgcacttgcgggcgcggcgcggaacagtgcggaaaggagcggtgcagacggcgcgcagtgacagtgggcgcaaagccgcgcagtaagcagcggcggggaacggtatacgcagtgccgcgggccgccgcacacagaagtatacgcgggccgaagtggggcgtcgcgcgcgggaagtgcggaatggcgggcaaggaaaggaggagacggaaagagggcgggaaagagagagagagagagtgaaaaaagaaagaaagaaagaaagaaagaaagaaagctcggagccacgccgcggggagagagagaaatgaaagcacggcacggcaaagcaaagcaaagcagacccagccagacccagccgagggaggagcgcgcgcaggacccgcgcggcgagcgagcgagcacggcgcgcgagcgagcgagcgagcgagcgcgcgagcgagcaaggcttgctgcgagcgatcgagcgagcgagcgggaaggatgagcgcgacccgcgcggcgacgaggacagcggcggcgctgtcctcggcgctgacgacgcctgtaaagcagcagcagcagcagcagctgcgcgtaggcgcggcgtcggcacggctggcggccgcggcgttctcgtccggcacgggcggagacgcggccaagaaggcggccgcggcgagggcgttctccacgggacgcggccccaacgcgacacgcgagaagagctcgctggccacggtccaggcggcgacggacgatgcgcgcttcgtcggcctgaccggcgcccaaatctttcatgagctcatgcgcgagcaccaggtggacaccatctttggctaccctggcggcgccattctgcccgtttttgatgccatttttgagagtgacgcgcttcaagttcattctcgctcgccacgagcagggcgccggccacatggccgagggctacgcgcgcgccacgggcaagcccggcgttgtcctcgtcacctcgggccctggagccaccaacaccatcaccccgatcatggatgcttacatggacggtacgccgctgctcgtgttcaccggccaggtgcagacctctgctgtcggcacggacgctttccaggagtgtgacattgttggcatcagccgcgcgtgcaccaagtggaacgtcatggtcaaggacgtgaaggagctcccgcgccgcatcaatgaggcctttgagattgccatgagcggccgcccgggtcccgtgctcgtcgatcttcctaaggatgtgaccgccgttgagctcaaggaaatgcccgacagctccccccaggttgctgtgcgccagaagcaaaaggtcgagcttttccacaaggagcgcattggcgctcctggcacggccgacttcaagctcattgccgagatgatcaaccgtgcggagcgacccgtcatctatgctggccagggtgtcatgcagagcccgttgaatggcccggctgtgctcaaggagttcgcggagaaggccaacattcccgtgaccaccaccatgcagggtctcggcggctttgacgagcgtagtcccctctccctcaagatgctcggcatgcacggctctgcctacgccaactactcgatgcagaacgccgatcttatcctggcgctcggtgcccgctttgatgatcgtgtgacgggccgcgttgacgcctttgctccggaggctcgccgtgccgagcgcgagggccgcggtggcatcgttcactttgagatttcccccaagaacctccacaaggtcgtccagcccaccgtcgcggtcctcggcgacgtggtcgagaacctcgccaacgtcacgccccacgtgcagcgccaggagcgcgagccgtggtttgcgcagatcgccgattggaaggagaagcacccttttctgctcgagtctgttgattcggacgacaaggttctcaagccgcagcaggtcctcacggagcttaacaagcagattctcgagattcaggagaaggacgccgaccaggaggtctacatcaccacgggcgtcggaagccaccagatgcaggcagcgcagttccttacctggaccaagccgcgccagtggatctcctcgggtggcgccggcactatgggctacggccttccctcggccattggcgccaagattgccaagcccgatgctattgttattgacatcgatggtgatgcttcttattcgatgaccggtatggaattgatcacagcagccgaattcaaggttggcgtgaagattcttcttttgcagaacaactttcagggcatggtcaagaacgttcaggatctcttttacgacaagcgctactcgggccaccgccatgttcaacccgcgcttcgacaaggtcgccgatgcgatgcgtgccaagggtctctactgcgcgaaacagtcggagctcaaggacaagatcaaggagtttctcgagtacgatgagggtcccgtcctcctcgaggttttcgtggacaaggacacgctcgtcttgcccatggtccccgctggctttccgctccacgagatggtcctcgagcctcctaagcccaaggacgcctaagttcttttttccatggcgggcgagcgagcgagcgcgcgagcgcgcaagtgcgcaagcgccttgccttgctttgcttcgcttcgctttgctttgcttcacacaacctaagtatgaattcaagttttcttgcttgtcggcgatgcctgcctgccaaccagccagccatccggccggccgtccttgacgccttcgcttccggcgcggccatcgattcaattcacccatccgatacgttccgccccctcacgtccgtctgcgcacgacccctgcacgaccacgccaaggccaacgcgccgctcagctcagcttgtcgacgagtcgcacgtcacatatctcagatgcatttggactgtgagtgttattatgccactagcacgcaacgatcttcggggtcctcgctcattgcatccgttcgggccctgcaggcgtggacgcgagtcgccgccgagacgctgcagcaggccgctccgacgcgagggctcgagctcgccgcgcccgcgcgatgtctgcctggcgccgactgatctctggagcgcaaggaagacacggcgacgcgaggaggaccgaagagagacgctggggtatgcaggatatacccggggcgggacattcgttccgcatacactcccccattcgagcttgctcgtccttggcagagccgagcgcgaacggttccgaacgcggcaaggattttggctctggtgggtggactccgatcgaggcgcaggttctccgcaggttctcgcaggccggcagtggtcgttagaaatagggagtgccggagtcttgacgcgccttagctcactctccgcccacgcgcgcatcgccgccatgccgccgtcccgtctgtcgctgcgctggccgcgaccggctgcgccagagtacgacagtgggacagagctcgaggcgacgcgaatcgctcgggttgtaagggtttcaagggtcgggcgtcgtcgcgtgccaaagtgaaaatagtaggggggggggggggtac

SEQ ID NO: 6 - DNA, 6175 bp, Artificial Sequence
  pCL0121
ctcttatctgcctcgcgccgttgaccgccgcttgactcttggcgcttgccgctcgcatcctgcctcgctcgcgcaggcgggcgggcgagtgggtgggtccgcagccttccgcgctcgcccgctagctcgctcgcgccgtgctgcagccagcagggcagcaccgcacggcaggcaggtcccggcgcggatcgatcgatccatcgatccatcgatccatcgatcgtgcggtcaaaaagaaaggaagaagaaaggaaaaagaaaggcgtgcgcacccgagtgcgcgctgagcgcccgctcgcggtcccgcggagcctccgcgttagtccccgccccgcgccgcgcagtcccccgggaggcatcgcgcacctctcgccgccccctcgcgcctcgccgattccccgcctccccttttccgcttcttcgccgcctccgctcgcggccgcgtcgcccgcgccccgctccctatctgctccccaggggggcactccgcaccttttgcgcccgctgccgccgccgcggccgccccgccgccctggtttcccccgcgagcgcggccgcgtcgccgcgcaaagactcgccgcgtgccgccccgagcaacgggtggcggcggcgcggcggcgggcggggcgcggcggcgcgtaggcggggctaggcgccggctaggcgaaacgccgcccccgggcgccgccgccgcccgctccagagcagtcgccgcgccagaccgccaacgcagagaccgagaccgaggtacgtcgcgcccgagcacgccgcgacgcgcggcagggacgaggagcacgacgccgcgccgcgccgcgcggggggggggagggagaggcaggacgcgggagcgagcgtgcatgtttccgcgcgagacgacgccgcgcgcgctggagaggagataaggcgcttggatcgcgagagggccagccaggctggaggcgaaaatgggtggagaggatagtatcttgcgtgcttggacgaggagactgacgaggaggacggatacgtcgatgatgatgtgcacagagaagaagcagttcgaaagcgactactagcaagcaagggatccatgaagttcgcgacctcggtcgcaattttgcttgtggccaacatagccaccgccctcgcgcagagcgatggctgcacccccaccgaccagacgatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctgggacacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagcaccaccatcaccaccactaacatatgagttatgagatccgaaagtgaaccttgtcctaacccgacagcgaatggcgggagggggcgggctaaaagatcgtattacatagtatttttcccctactctttgtgtttgtcttttttttttttttgaacgcattcaagccacttgtctgggtttacttgtttgtttgcttgcttgcttgcttgcttgcctgcttcttggtcagacggcccaaaaaagggaaaaaattcattcatggcacagataagaaaaagaaaaagtttgtcgaccaccgtcatcagaaagcaagagaagagaaacactcgcgctcacattctcgctcgcgtaagaatcttagccacgcatacgaagtaatttgtccatctggcgaatctttacatgagcgttttcaagctggagcgtgagatcatacctttcttgatcgtaatgttccaaccttgcataggcctcgttgcgatccgctagcaatgcgtcgtactcccgttgcaactgcgccatcgcctcattgtgacgtgagttcagattcttctcgagaccttcgagcgctgctaatttcgcctgacgctccttcttttgtgcttccatgacacgccgcttcaccgtgcgttccacttcttcctcagacatgcccttggctgcctcgacctgctcggtaaaacgggccccagcacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatacatggtcgacctgcaggaacctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccaagcttccaattttaggccccccactgaccgaggtctgtcgataatccacttttccattgattttccaggtttcgttaactcatgccactgagcaaaacttcggtctttcctaacaaaagctctcctcacaaagcatggcgcggcaacggacgtgtcctcatactccactgccacacaaggtcgataaactaagctcctcacaaatagaggagaattccactgacaactgaaaacaatgtatgagagacgatcaccactggagcggcgcggcggttgggcgcggaggtcggcagcaaaaacaagcgactcgccgagcaaacccgaatcagccttcagacggtcgtgcctaacaacacgccgttctaccccgccttcttcgcgccccttcgcgtccaagcatccttcaagtttatctctctagttcaacttcaagaagaacaacaccaccaacaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcggtac

SEQ ID NO: 7 - DNA, 7174 bp, Artificial Sequence
  pCL0125
ctcttatctgcctcgcgccgttgaccgccgcttgactcttggcgcttgccgctcgcatcctgcctcgctcgcgcaggcgggcgggcgagtgggtgggtccgcagccttccgcgctcgcccgctagctcgctcgcgccgtgctgcagccagcagggcagcaccgcacggcaggcaggtcccggcgcggatcgatcgatccatcgatccatcgatccatcgatcgtgcggtcaaaaagaaaggaagaagaaaggaaaaagaaaggcgtgcgcacccgagtgcgcgctgagcgcccgctcgcggtcccgcggagcctccgcgttagtccccgccccgcgccgcgcagtcccccgggaggcatcgcgcacctctcgccgccccctcgcgcctcgccgattccccgcctccccttttccgcttcttcgccgcctccgctcgcggccgcgtcgcccgcgccccgctccctatctgctccccaggggggcactccgcaccttttgcgcccgctgccgccgccgcggccgccccgccgccctggtttcccccgcgagcgcggccgcgtcgccgcgcaaagactcgccgcgtgccgccccgagcaacgggtggcggcggcgcggcggcgggcggggcgcggcggcgcgtaggcggggctaggcgccggctaggcgaaacgccgcccccgggcgccgccgccgcccgctccagagcagtcgccgcgccagaccgccaacgcagagaccgagaccgaggtacgtcgcgcccgagcacgccgcgacgcgcggcagggacgaggagcacgacgccgcgccgcgccgcgcggggggggggagggagaggcaggacgcgggagcgagcgtgcatgtttccgcgcgagacgacgccgcgcgcgctggagaggagataaggcgcttggatcgcgagagggccagccaggctggaggcgaaaatgggtggagaggatagtatcttgcgtgcttggacgaggagactgacgaggaggacggatacgtcgatgatgatgtgcacagagaagaagcagttcgaaagcgactactagcaagcaagggatccatgaagaacatcatctcgctcgtctccaagaagaaggccgcttccaagaacgaggacaagaacatctcggagtcgtcccgcgacatcgtcaaccagcaggaggtgtttaacactgaggactttgaggagggaaagaaggattccgcttttgagcttgatcacctcgagtttaccaccaactcggcccagctcggtgatagcgacgaggacaacgagaacgtcatcaacgagatgaacgcgacggacgatgctaacgaggctaacagcgaggagaagtccatgaccctcaagcaggccctccttaagtacccgaaggccgccctttggtccatccttgtttccaccactcttgtcatggagggttacgacaccgcccttctcagcgccctctacgctctccctgtttttcagcgcaagtttggcaccctcaacggagagggctcctacgagatcacctcccagtggcagatcggtcttaacatgtgcgtgctctgcggtgagatgatcggtctccagatcaccacctacatggttgagtttatgggtaaccgctacactatgattaccgctctcggcctcctcacggcgtacatctttatcctctactactgcaagtcccttgccatgattgccgtcggtcagattctcagcgccattccctggggttgcttccagtccctcgccgtcacctacgcctccgaggtctgccccctcgcccttcgttactacatgacctcctacagcaacatttgctggctgtttggacagatttttgcttccggcatcatgaagaactcccaggagaacctcggcaacagcgatctcggttacaagctccccttcgcgctccagtggatttggccggcccccctcatgatcggcattttctttgcccccgagagcccctggtggctcgtgcgtaaggatcgtgtcgccgaggcccgcaagtcgctctcccgtattctctccggcaagggcgccgagaaggacattcaggtcgacctcaccctcaagcagattgagcttaccattgagaaggagcgcctcctcgcgtccaagtcgggctccttttttaactgctttaagggagtcaacggccgccgtacccgtcttgcctgcctgacctgggttgcccagaactcgtcgggcgccgtgctcctcggctactccacctacttttttgagaagaagcaggtcatggccaccgacaaggctttcactttctcgcttatccagtactgcctcggccttgccggtactctctgcagctgggtcatctcgggccgcgtgggccgctggaccatcctcacgtacggcctcgcttttcagatggtttgcctctttattatcggtggcatgggatttggttcgggctcctccgcttccaacggcgccggcggcctccttctcgctctcagctttttttacaacgccggaattggtgccgtcgtctactgcattgttgccgagatcccttcggccgagctccgcacgaagaccatcgtccttgcccgcatctgctacaacctcatggccgtcatcaacgccatcctcaccccctacatgctcaacgtgtcggactggaactggggcgccaagacgggactttactggggtggctttaccgctgtgacccttgcctgggttattatcgacctccccgagaccaccggccgtaccttctccgagatcaacgagctctttaaccagggtgtccccgcccgtaagtttgcctcgacggtcgtcgacccttttggaaagggcaagacgcagcacgactccctcgccgacgagtccatttcgcagtccagctcgatcaagcagcgtgagcttaacgccgccgacaagtgctaacatatgagttatgagatccgaaagtgaaccttgtcctaacccgacagcgaatggcgggagggggcgggctaaaagatcgtattacatagtatttttcccctactctttgtgtttgtcttttttttttttttgaacgcattcaagccacttgtctgggtttacttgtttgtttgcttgcttgcttgcttgcttgcctgcttcttggtcagacggcccaaaaaagggaaaaaattcattcatggcacagataagaaaaagaaaaagtttgtcgaccaccgtcatcagaaagcaagagaagagaaacactcgcgctcacattctcgctcgcgtaagaatcttagccacgcatacgaagtaatttgtccatctggcgaatctttacatgagcgttttcaagctggagcgtgagatcatacctttcttgatcgtaatgttccaaccttgcataggcctcgttgcgatccgctagcaatgcgtcgtactcccgttgcaactgcgccatcgcctcattgtgacgtgagttcagattcttctcgagaccttcgagcgctgctaatttcgcctgacgctccttcttttgtgcttccatgacacgccgcttcaccgtgcgttccacttcttcctcagacatgcccttggctgcctcgacctgctcggtaaaacgggccccagcacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatacatggtcgacctgcaggaacctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccaagcttgaggtctgtcgataatccacttttccattgattttccaggtttcgttaactcatgccactgagcaaaacttcggtctttcctaacaaaagctctcctcacaaagcatggcgcggcaacggacgtgtcctcatactccactgccacacaaggtcgataaactaagctcctcacaaatagaggagaattccactgacaactgaaaacaatgtatgagagacgatcaccactggagcggcgcggcggttgggcgcggaggtcggcagcaaaaacaagcgactcgccgagcaaacccgaatcagccttcagacggtcgtgcctaacaacacgccgttctaccccgccttcttcgcgccccttcgcgtccaagcatccttcaagtttatctctctagttcaacttcaagaagaacaacaccaccaacaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcggtac

SEQ ID NO: 8 - DNA, 6871 bp, Artificial Sequence
  pCL0126
ctcttatctgcctcgcgccgttgaccgccgcttgactcttggcgcttgccgctcgcatcctgcctcgctcgcgcaggcgggcgggcgagtgggtgggtccgcagccttccgcgctcgcccgctagctcgctcgcgccgtgctgcagccagcagggcagcaccgcacggcaggcaggtcccggcgcggatcgatcgatccatcgatccatcgatccatcgatcgtgcggtcaaaaagaaaggaagaagaaaggaaaaagaaaggcgtgcgcacccgagtgcgcgctgagcgcccgctcgcggtcccgcggagcctccgcgttagtccccgccccgcgccgcgcagtcccccgggaggcatcgcgcacctctcgccgccccctcgcgcctcgccgattccccgcctccccttttccgcttcttcgccgcctccgctcgcggccgcgtcgcccgcgccccgctccctatctgctccccaggggggcactccgcaccttttgcgcccgctgccgccgccgcggccgccccgccgccctggtttcccccgcgagcgcggccgcgtcgccgcgcaaagactcgccgcgtgccgccccgagcaacgggtggcggcggcgcggcggcgggcggggcgcggcggcgcgtaggcggggctaggcgccggctaggcgaaacgccgcccccgggcgccgccgccgcccgctccagagcagtcgccgcgccagaccgccaacgcagagaccgagaccgaggtacgtcgcgcccgagcacgccgcgacgcgcggcagggacgaggagcacgacgccgcgccgcgccgcgcggggggggggagggagaggcaggacgcgggagcgagcgtgcatgtttccgcgcgagacgacgccgcgcgcgctggagaggagataaggcgcttggatcgcgagagggccagccaggctggaggcgaaaatgggtggagaggatagtatcttgcgtgcttggacgaggagactgacgaggaggacggatacgtcgatgatgatgtgcacagagaagaagcagttcgaaagcgactactagcaagcaagggatccatggagaacggcactaagcgtgagggccttggaaagctcactgtcagctcctccctccaggtcgagcagcccctcgcccccagcaagctctggaagattattgtcgtcgcttcgatcgccgccggtgtccagtttggctgggcccttcagctctcgctccttactccctacgttcagcttctcggtattcctcacaagttcgcgtcctttatttggctctgcggaccgatttcgggtatgatcgtgcagccggtcgtcggttactactcggataactgctcgtcgcgttttggccgtcgccgtccttttatcgccgctggtgccgctctcgttatgattgccgttttccttatcggctttgccgctgacctcggccacgcctcgggtgacaccctcggtaagggtttcaagcctcgcgccattgccgtcttcgtcgtcggcttctggattctcgacgtcgctaacaacatgcttcagggaccctgccgcgccctcctcgctgacctctccggcggaaagtccggccgcatgcgcaccgctaacgccttcttcagcttcttcatggccgtcggcaacatcctcggctacgccgccggctcctacagccacctctttaaggtctttcccttttccaagacgaaggcctgcgatatgtactgcgctaacctcaagtcctgcttttttatcgccatcttccttcttctctcgctcacgaccattgccctgaccctcgtccgcgagaacgagctccctgagaaggacgagcaggagatcgacgagaagctcgctggcgccggcaagtccaaggtcccgttcttcggcgagatctttggcgccctcaaggagcttccccgcccgatgtggattctcctccttgttacttgcctcaactggattgcctggtttccctttttcctctacgacactgactggatggctaaggaggtttttggtggccaggtcggtgatgcccgcctctacgatctcggcgtccgcgctggtgcgatgggccttctccttcagagcgtcgtcctcggctttatgtccctcggagttgagtttctcggtaagaagatcggcggagccaagcgcctttggggcatcctcaactttgtgctcgccatctgcctggctatgaccatccttgtgactaagatggctgagaagtcccgccagcatgatcctgccggcacgcttatgggtccgacccccggcgtcaagatcggcgctcttcttctctttgctgccctcggtattcccctcgccgccactttttcgattcccttcgctctcgcctcgattttctcctccaaccgtggttcgggtcagggtctcagcctcggtgtgctcaaccttgccattgtggtcccccagatgcttgtctcgcttgtcggcggaccctgggatgatcttttcggcggaggaaaccttccgggcttcgttgttggcgctgtcgccgccgcggcctcggctgtcctcgccctcaccatgctcccttcgcctcccgccgatgccaagcctgccgttgccatgggccttagcattaagtaacatatgagttatgagatccgaaagtgaaccttgtcctaacccgacagcgaatggcgggagggggcgggctaaaagatcgtattacatagtatttttcccctactctttgtgtttgtcttttttttttttttgaacgcattcaagccacttgtctgggtttacttgtttgtttgcttgcttgcttgcttgcttgcctgcttcttggtcagacggcccaaaaaagggaaaaaattcattcatggcacagataagaaaaagaaaaagtttgtcgaccaccgtcatcagaaagcaagagaagagaaacactcgcgctcacattctcgctcgcgtaagaatcttagccacgcatacgaagtaatttgtccatctggcgaatctttacatgagcgttttcaagctggagcgtgagatcatacctttcttgatcgtaatgttccaaccttgcataggcctcgttgcgatccgctagcaatgcgtcgtactcccgttgcaactgcgccatcgcctcattgtgacgtgagttcagattcttctcgagaccttcgagcgctgctaatttcgcctgacgctccttcttttgtgcttccatgacacgccgcttcaccgtgcgttccacttcttcctcagacatgcccttggctgcctcgacctgctcggtaaaacgggccccagcacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatacatggtcgacctgcaggaacctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccaagcttgaggtctgtcgataatccacttttccattgattttccaggtttcgttaactcatgccactgagcaaaacttcggtctttcctaacaaaagctctcctcacaaagcatggcgcggcaacggacgtgtcctcatactccactgccacacaaggtcgataaactaagctcctcacaaatagaggagaattccactgacaactgaaaacaatgtatgagagacgatcaccactggagcggcgcggcggttgggcgcggaggtcggcagcaaaaacaagcgactcgccgagcaaacccgaatcagccttcagacggtcgtgcctaacaacacgccgttctaccccgccttcttcgcgccccttcgcgtccaagcatccttcaagtttatctctctagttcaacttcaagaagaacaacaccaccaacaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcggtac

SEQ ID NO: 9 - DNA, 6611 bp, Artificial Sequence
  pCL0122
ctcttatctgcctcgcgccgttgaccgccgcttgactcttggcgcttgccgctcgcatcctgcctcgctcgcgcaggcgggcgggcgagtgggtgggtccgcagccttccgcgctcgcccgctagctcgctcgcgccgtgctgcagccagcagggcagcaccgcacggcaggcaggtcccggcgcggatcgatcgatccatcgatccatcgatccatcgatcgtgcggtcaaaaagaaaggaagaagaaaggaaaaagaaaggcgtgcgcacccgagtgcgcgctgagcgcccgctcgcggtcccgcggagcctccgcgttagtccccgccccgcgccgcgcagtcccccgggaggcatcgcgcacctctcgccgccccctcgcgcctcgccgattccccgcctccccttttccgcttcttcgccgcctccgctcgcggccgcgtcgcccgcgccccgctccctatctgctccccaggggggcactccgcaccttttgcgcccgctgccgccgccgcggccgccccgccgccctggtttcccccgcgagcgcggccgcgtcgccgcgcaaagactcgccgcgtgccgccccgagcaacgggtggcggcggcgcggcggcgggcggggcgcggcggcgcgtaggcggggctaggcgccggctaggcgaaacgccgcccccgggcgccgccgccgcccgctccagagcagtcgccgcgccagaccgccaacgcagagaccgagaccgaggtacgtcgcgcccgagcacgccgcgacgcgcggcagggacgaggagcacgacgccgcgccgcgccgcgcggggggggggagggagaggcaggacgcgggagcgagcgtgcatgtttccgcgcgagacgacgccgcgcgcgctggagaggagataaggcgcttggatcgcgagagggccagccaggctggaggcgaaaatgggtggagaggatagtatcttgcgtgcttggacgaggagactgacgaggaggacggatacgtcgatgatgatgtgcacagagaagaagcagttcgaaagcgactactagcaagcaagggatccatgaagttcgcgacctcggtcgcaattttgcttgtggccaacatagccaccgccctcgcgcagagcgatggctgcacccccaccgaccagacgatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctgggacacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagcaccaccatcaccaccactaacatatgagttatgagatccgaaagtgaaccttgtcctaacccgacagcgaatggcgggagggggcgggctaaaagatcgtattacatagtatttttcccctactctttgtgtttgtcttttttttttttttgaacgcattcaagccacttgtctgggtttacttgtttgtttgcttgcttgcttgcttgcttgcctgcttcttggtcagacggcccaaaaaagggaaaaaattcattcatggcacagataagaaaaagaaaaagtttgtcgaccaccgtcatcagaaagcaagagaagagaaacactcgcgctcacattctcgctcgcgtaagaatcttagccacgcatacgaagtaatttgtccatctggcgaatctttacatgagcgttttcaagctggagcgtgagatcatacctttcttgatcgtaatgttccaaccttgcataggcctcgttgcgatccgctagcaatgcgtcgtactcccgttgcaactgcgccatcgcctcattgtgacgtgagttcagattcttctcgagaccttcgagcgctgctaatttcgcctgacgctccttcttttgtgcttccatgacacgccgcttcaccgtgcgttccacttcttcctcagacatgcccttggctgcctcgacctgctcggtaaaacgggccccagcacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatacatggtcgacctgcaggaacctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccaagcttccaattttaggccccccactgaccgaggtctgtcgataatccacttttccattgattttccaggtttcgttaactcatgccactgagcaaaacttcggtctttcctaacaaaagctctcctcacaaagcatggcgcggcaacggacgtgtcctcatactccactgccacacaaggtcgataaactaagctcctcacaaatagaggagaattccactgacaactgaaaacaatgtatgagagacgatcaccactggagcggcgcggcggttgggcgcggaggtcggcagcaaaaacaagcgactcgccgagcaaacccgaatcagccttcagacggtcgtgcctaacaacacgccgttctaccccgccttcttcgcgccccttcgcgtccaagcatccttcaagtttatctctctagttcaacttcaagaagaacaacaccaccaacaccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgaattcccggggtac

SEQ ID NO: 10 - DNA, 7295 bp, Artificial Sequence
  pCL0127
ctcttatctgcctcgcgccgttgaccgccgcttgactcttggcgcttgccgctcgcatcctgcctcgctcgcgcaggcgggcgggcgagtgggtgggtccgcagccttccgcgctcgcccgctagctcgctcgcgccgtgctgcagccagcagggcagcaccgcacggcaggcaggtcccggcgcggatcgatcgatccatcgatccatcgatccatcgatcgtgcggtcaaaaagaaaggaagaagaaaggaaaaagaaaggcgtgcgcacccgagtgcgcgctgagcgcccgctcgcggtcccgcggagcctccgcgttagtccccgccccgcgccgcgcagtcccccgggaggcatcgcgcacctctcgccgccccctcgcgcctcgccgattccccgcctccccttttccgcttcttcgccgcctccgctcgcggccgcgtcgcccgcgccccgctccctatctgctccccaggggggcactccgcaccttttgcgcccgctgccgccgccgcggccgccccgccgccctggtttcccccgcgagcgcggccgcgtcgccgcgcaaagactcgccgcgtgccgccccgagcaacgggtggcggcggcgcggcggcgggcggggcgcggcggcgcgtaggcggggctaggcgccggctaggcgaaacgccgcccccgggcgccgccgccgcccgctccagagcagtcgccgcgccagaccgccaacgcagagaccgagaccgaggtacgtcgcgcccgagcacgccgcgacgcgcggcagggacgaggagcacgacgccgcgccgcgccgcgcggggggggggagggagaggcaggacgcgggagcgagcgtgcatgtttccgcgcgagacgacgccgcgcgcgctggagaggagataaggcgcttggatcgcgagagggccagccaggctggaggcgaaaatgggtggagaggatagtatcttgcgtgcttggacgaggagactgacgaggaggacggatacgtcgatgatgatgtgcacagagaagaagcagttcgaaagcgactactagcaagcaagggatccatgaccaacgagacctcggaccgccctctcgtgcactttacccccaacaagggttggatgaacgatcccaacggcctctggtacgacgagaaggatgctaagtggcacctttactttcagtacaaccctaacgacaccgtctggggcaccccgctcttctggggccacgccacctccgacgacctcaccaactgggaggaccagcccattgctatcgcccccaagcgcaacgactcgggagctttttccggttccatggttgtggactacaacaacacctccggtttttttaacgacaccattgacccccgccagcgctgcgtcgccatctggacctacaacacgcccgagagcgaggagcagtacatcagctacagccttgatggaggctacacctttaccgagtaccagaagaaccctgtcctcgccgccaactccacccagttccgcgaccctaaggttttttggtacgagccttcccagaagtggattatgaccgccgctaagtcgcaggattacaagatcgagatctacagcagcgacgacctcaagtcctggaagcttgagtccgcctttgccaacgagggttttctcggataccagtacgagtgccccggtctcatcgaggtccccaccgagcaggacccgtccaagtcctactgggtcatgtttatttccatcaaccctggcgcccctgccggcggcagcttcaaccagtacttcgtcggctcctttaacggcacgcattttgaggccttcgacaaccagtcccgcgtcgtcgacttcggcaaggactactacgccctccagaccttctttaacaccgaccccacctacggcagcgccctcggtattgcttgggcctccaactgggagtactccgctttcgtccccactaacccctggcgcagctcgatgtccctcgtccgcaagttttcgcttaacaccgagtaccaggccaaccccgagaccgagcttattaacctgaaggccgagcctattctcaacatctccaacgctggcccctggtcccgctttgctactaacactaccctcaccaaggccaactcctacaacgtcgatctctccaactccaccggtactcttgagtttgagctcgtctacgccgtcaacaccacccagaccatctccaagtccgtcttcgccgacctctccctctggttcaagggccttgaggaccccgaggagtacctgcgcatgggttttgaggtctccgcctcctccttcttcctcgatcgcggtaactccaaggttaagtttgtcaaggagaacccctactttactaaccgtatgagcgtcaacaaccagccctttaagtccgagaacgatcttagctactacaaggtttacggcctcctcgaccagaacattctcgagctctactttaacgacggagatgtcgtcagcaccaacacctactttatgaccactggaaacgccctcggcagcgtgaacatgaccaccggagtcgacaacctcttttacattgacaagtttcaggttcgcgaggttaagtaacatatgagttatgagatccgaaagtgaaccttgtcctaacccgacagcgaatggcgggagggggcgggctaaaagatcgtattacatagtatttttcccctactctttgtgtttgtcttttttttttttttgaacgcattcaagccacttgtctgggtttacttgtttgtttgcttgcttgcttgcttgcttgcctgcttcttggtcagacggcccaaaaaagggaaaaaattcattcatggcacagataagaaaaagaaaaagtttgtcgaccaccgtcatcagaaagcaagagaagagaaacactcgcgctcacattctcgctcgcgtaagaatcttagccacgcatacgaagtaatttgtccatctggcgaatctttacatgagcgttttcaagctggagcgtgagatcatacctttcttgatcgtaatgttccaaccttgcataggcctcgttgcgatccactagcaatgcgtcgtactcccgttgcagctgcgccatcgcctcattgtgacgtgagttcagattcttctcgagaccttcgagcgctgctaatttcgcctgacgctccttcttttgtgcttccatgacacgccgcttcaccgtgcgttccacttcttcctcagacatgcccttggctgcctcgacctgctcggtaaaacgggccccagcacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatacatggtcgacctgcaggaacctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccaagcttgaggtctgtcgataatccacttttccattgattttccaggtttcgttaactcatgccactgagcaaaacttcggtctttcctaacaaaagctctcctcacaaagcatggcgcggcaacggacgtgtcctcatactccactgccacacaaggtcgataaactaagctcctcacaaatagaggagaattccactgacaactgaaaacaatgtatgagagacgatcaccactggagcggcgcggcggttgggcgcggaggtcggcagcaaaaacaagcgactcgccgagcaaacccgaatcagccttcagacggtcgtgcctaacaacacgccgttctaccccgccttcttcgcgccccttcgcgtccaagcatccttcaagtttatctctctagttcaacttcaagaagaacaacaccaccaacaccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgaattcccggggtac

(19) United States
(12) Patent Application Publication
(10) Pub. No.: US 2011/0195449 A1
(43) Pub. Date: Aug. 11, 2011

(54) Recombinant Thraustochytrids that Grow on Sucrose, and Compositions, Methods of Making, and Uses Thereof

(75) Inventors: James Casey Lippmeier, Columbia, MD (US); Kirk Emil Apt, Ellicott City, MD (US); Jon Milton Hansen, West Friendship, MD (US)

(73) Assignee: Martek Biosciences Corporation

(21) Appl. No.: 12/980,322

(22) Filed: Dec. 28, 2010

Related U.S. Application Data
(60) Provisional application No. 61/290,443, filed on Dec. 28, 2009.

(51) Int. Cl. C12P 1/00; C12N 15/63; C12N 1/13

(52) U.S. Cl. 435/41; 435/471; 435/257.2

(57) ABSTRACT

The present invention is directed to recombinant thraustochytrids that grow on sucrose and cell cultures comprising the recombinant thraustochytrids as well as methods of producing cell cultures, biomasses, microbial oils, compositions, and biofuels using the recombinant thraustochytrids.

### CROSS-REFERENCE TO RELATED APPLICATIONS

This application claims the benefit of the filing date of U.S. Appl. No. 61/290,443, filed Dec. 28, 2009, which is hereby incorporated by reference herein in its entirety.

### REFERENCE TO SEQUENCE LISTING SUBMITTED ELECTRONICALLY

The content of the electronically submitted sequence listing (“sequence listing_ascii.txt”, 77,186 bytes, created on Dec. 23, 2010) filed with the application is incorporated herein by reference in its entirety.

### BACKGROUND OF THE INVENTION

1. Field of the Invention

The present invention is directed to recombinant thraustochytrids that grow on sucrose and cell cultures comprising the recombinant thraustochytrids, as well as methods of producing cell cultures, biomasses, microbial oils, compositions, and biofuels using the recombinant thraustochytrids.

2. Background

Fatty acids are classified based on the length and saturation characteristics of the carbon chain. Fatty acids are termed short chain, medium chain, or long chain fatty acids based on the number of carbons present in the chain, are termed saturated fatty acids when no double bonds are present between the carbon atoms, and are termed unsaturated fatty acids when double bonds are present. Unsaturated long chain fatty acids are monounsaturated when only one double bond is present and are polyunsaturated when more than one double bond is present.

Polyunsaturated fatty acids (PUFAs) are classified based on the position of the first double bond from the methyl end of the fatty acid: omega-3 (n-3) fatty acids contain a first double bond at the third carbon, while omega-6 (n-6) fatty acids contain a first double bond at the sixth carbon. For example, docosahexaenoic acid (“DHA”) is an omega-3 long chain polyunsaturated fatty acid (LC-PUFA) with a chain length of 22 carbons and 6 double bonds, often designated as “22:6 n-3.” Other omega-3 LC-PUFAs include eicosapentaenoic acid (“EPA”), designated as “20:5 n-3,” and omega-3 docosapentaenoic acid (“DPA n-3”), designated as “22:5 n-3.” DHA and EPA have been termed “essential” fatty acids. Omega-6 LC-PUFAs include arachidonic acid (“ARA”), designated as “20:4 n-6,” and omega-6 docosapentaenoic acid (“DPA n-6”), designated as “22:5 n-6.”

Omega-3 fatty acids are biologically important molecules that affect cellular physiology due to their presence in cell membranes, regulate production and gene expression of biologically active compounds, and serve as biosynthetic substrates. Roche, H. M., Proc. Nutr. Soc. 58: 397-401 (1999). DHA, for example, accounts for approximately 15%-20% of lipids in the human cerebral cortex and 30%-60% of lipids in the retina, is concentrated in the testes and sperm, and is an important component of breast milk. Jean-Pascal Bergé & Gilles Barnathan, Fatty Acids from Lipids of Marine Organisms: Molecular Biodiversity, Roles as Biomarkers, Biologically Active Compounds, and Economical Aspects, in Marine Biotechnology I 49 (T. Scheper, ed., 2005). DHA accounts for up to 97% of the omega-3 fatty acids in the brain and up to 93% of the omega-3 fatty acids in the retina. Moreover, DHA is essential for both fetal and infant development as well as maintenance of cognitive functions in adults. Id. Omega-3 fatty acids, including DHA and EPA, also possess anti-inflammatory properties. See, e.g., id. and Simopoulos, A. P., J. Am. Coll. Nutr. 21: 495-595 (2002). In particular, EPA competes with arachidonic acid for synthesis of eicosanoids such as prostaglandins and leukotrienes through cyclooxygenases and lipoxygenases. Id. Because omega-3 fatty acids are not synthesized de novo in the human body, these fatty acids must be derived from nutritional sources.

Thraustochytrids, which are microalgal organisms of the order Thraustochytriales, are recognized as alternative sources for the production of lipids. For example, strains of thraustrochytrid species have been reported to produce omega-3 fatty acids as a high percentage of the total fatty acids produced by the organisms. See, e.g., U.S. Pat. No. 5,130,242; Huang, J. et al., J. Am. Oil. Chem. Soc. 78: 605-610 (2001); and Huang, J. et al., Mar. Biotechnol. 5: 450-457 (2003), incorporated by reference herein in their entireties. Thraustochytrids have also been recognized as sources of lipids for the production of biofuels. See, e.g., U.S. Publ. No. 2009/0064567 and WO 2008/067605, incorporated by reference herein in their entireties.

While thraustochytrids metabolize glucose and/or fructose, which are the two sugar components of sucrose, they do not appear to naturally metabolize sucrose. See, e.g., Aki et al., JAOCS 80:789-794 (2003); Yokochi et al., Appl. Microbiol. Biotechnol. 49:72-76 (1998); Honda et al., Mycol. Res. 4:439-448 (1998); Singh, World J. of Microbiology 12:76-81 (1996); Goldstein, American J. of Botany 50:271-279 (1963); and U.S. Pat. No. 5,340,742. As such, thraustochytrid cultures, including commercial and industrial cultures, currently require glucose syrups as carbon and energy sources, rather than more inexpensive carbohydrate sources containing sucrose.

A continuing need exists for producing thraustochytrids with the ability to grow on alternative carbon sources such as sucrose for use in commercial and industrial applications.

### BRIEF SUMMARY OF THE INVENTION

The present invention is directed to a method of producing a thraustochytrid cell culture, comprising: (a) transforming a thraustochytrid cell with a nucleic acid molecule comprising a polynucleotide sequence encoding a heterologous sucrase, and (b) growing the transformed thraustochytrid cell in a culture medium comprising sucrose as a carbon source. In some embodiments, the sucrase is an invertase. In some embodiments, the sucrase is operably linked to a signal peptide. In some embodiments, the polynucleotide sequence encoding the heterologous sucrase is at least 90% identical to a sequence selected from the group consisting of: a polynucleotide sequence encoding the amino acid sequence of Accession No. P00724 or S33920; a polynucleotide sequence encoding the amino acid sequence of Accession No. P00724 or S33920, wherein the polynucleotide sequence is codon-optimized for expression in Schizochytrium; and the polynucleotide sequence of SEQ ID NO: 1 or SEQ ID NO: 4. In some embodiments, the method further comprises transforming the thraustochytrid cell with a nucleic acid molecule comprising a polynucleotide sequence encoding a heterologous sucrose transporter. In some embodiments, the polynucleotide sequence encoding the heterologous sucrose transporter is at least 90% identical to a sequence selected from the group consisting of: a polynucleotide sequence of Accession No. L47346 or X69165; and a polynucleotide sequence of Accession No. L47346 or X69165 that is codon-optimized for expression in Schizochytrium. In some embodiments, the thraustochytrid is a Schizochytrium or a Thraustochytrium. In some embodiments, the invention is directed to a thraustochytrid cell culture produced by the method.

The present invention is also directed to a thraustochytrid cell comprising a nucleic acid molecule comprising a polynucleotide sequence encoding a heterologous sucrase. In some embodiments, the sucrase is an invertase. In some embodiments, the sucrase is operably linked to a signal peptide. In some embodiments, the polynucleotide sequence encoding the heterologous sucrase is at least 90% identical to a sequence selected from the group consisting of: a polynucleotide sequence encoding the amino acid sequence of Accession No. P00724 or S33920; a polynucleotide sequence encoding the amino acid sequence of Accession No. P00724 or S33920, wherein the polynucleotide sequence is codon-optimized for expression in Schizochytrium; and the polynucleotide sequence of SEQ ID NO: 1 or SEQ ID NO: 4. In some embodiments, the cell further comprises a polynucleotide sequence encoding a heterologous sucrose transporter. In some embodiments, the polynucleotide sequence encoding the heterologous sucrose transporter is at least 90% identical to a sequence selected from the group consisting of: a polynucleotide sequence of Accession No. L47346 or X69165; and a polynucleotide sequence of Accession No. L47346 or X69165 that is codon-optimized for expression in Schizochytrium. In some embodiments, the thraustochytrid is a Schizochytrium or a Thraustochytrium.

The present invention is also directed to a thraustochytrid culture comprising: (a) any of the thraustochytrid cells described herein, and (b) a cell culture medium comprising sucrose as a carbon source.

The present invention is also directed to a method of producing a thraustochytrid biomass, comprising: (a) growing any of the thraustochytrid cells described herein in a culture medium comprising sucrose as a carbon source, and (b) harvesting the biomass from the culture medium.

The present invention is also directed to a method of producing a microbial oil, comprising: (a) growing any of the thraustochytrid cells described herein in a culture medium comprising sucrose as a carbon source to produce a biomass, and (b) extracting an oil from the biomass.

The present invention is also directed to a method of producing a food product, cosmetic, industrial composition, or pharmaceutical composition for an animal or human, comprising: (a) growing any of the thraustochytrid cells described herein in a culture medium comprising sucrose as a carbon source, (b) harvesting a biomass from the culture medium, and (c) preparing the food product, cosmetic, industrial composition, or pharmaceutical composition from the biomass. In some embodiments, the method further comprises extracting an oil from the biomass and preparing the food product, cosmetic, industrial composition, or pharmaceutical composition from the oil. In some embodiments, the food product is an infant formula. In some embodiments, the infant formula is suitable for premature infants. In some embodiments, the food product is milk, a beverage, a therapeutic drink, a nutritional drink, or a combination thereof. In some embodiments, the food product is an additive for animal or human food. In some embodiments, the food product is a nutritional supplement. In some embodiments, the food product is an animal feed. In some embodiments, the animal feed is an aquaculture feed. In some embodiments, the animal feed is a domestic animal feed, a zoological animal feed, a work animal feed, a livestock feed, or a combination thereof.

The present invention is also directed to a method for producing a biofuel, comprising: (a) growing any of the thraustochytrid cells described herein in a culture medium comprising sucrose as a carbon source to produce a biomass, (b) extracting an oil from the biomass, and (c) producing a biofuel by transesterifying the oil, cracking the oil, processing the oil by thermal depolymerization, adding the oil to a petroleum refining process, or a combination thereof. In some embodiments, the biofuel is produced by transesterifying the oil. In some embodiments, the biofuel is a biodiesel. In some embodiments, the biofuel is produced by cracking the oil. In some embodiments, the biofuel is a jet biofuel. In some embodiments, the biofuel is produced by processing the oil by thermal depolymerization. In some embodiments, the biofuel is a renewable diesel. In some embodiments, the biofuel is produced by adding the oil to a petroleum refining process. In some embodiments, the biofuel is a co-processed renewable diesel.

### BRIEF DESCRIPTION OF THE DRAWINGS/FIGURES

FIG. 1 shows a codon usage table for Schizochytrium.

FIG. 2 shows a plasmid map of pSchiz-EPCT(+)-s1Suc2_CL0076, also termed pCL0076 (SEQ ID NO:2).

FIG. 3 shows dry weight (g/L) of cell pellets from cultures of Schizochytrium sp. ATCC 20888 transformed with pCL0076 grown on sucrose-SSFM. The transformants are referred to as 1-1, 1-3, 1-24, 3-1, 3-2, 3-5, 3-21, 4-1, 4-24, and 4-31. “Control” refers to wild-type Schizochytrium sp. ATCC 20888 cells grown on glucose-SSFM.

FIG. 4 shows fat content (expressed as % weight of the dry biomass) in cell pellets from cultures of Schizochytrium sp. ATCC 20888 transformed with pCL0076 grown on sucrose-SSFM. The transformants are referred to as 1-1, 1-3, 1-24, 3-1, 3-2, 3-5, 3-21, 4-1, 4-24, and 4-31. “Control” refers to wild-type Schizochytrium sp. ATCC 20888 cells grown on glucose-SSFM.

FIG. 5 shows dry weight (g/L) of cell pellets measured over time (days) for cultures of Schizochytrium sp. ATCC 20888 transformed with pCL0076 grown on sucrose-SSFM. The transformants are referred to as 1-3 and 3-5. “Control” refers to wild-type Schizochytrium sp. ATCC 20888 cells grown on glucose-SSFM.

FIG. 6 shows fat content (expressed as % weight of the dry biomass) in cell pellets from cultures of two transformants grown on sucrose-SSFM. The transformants are referred to as 1-3 and 3-5. “Control” refers to wild-type Schizochytrium sp. ATCC 20888 cells grown on glucose-SSFM.

FIG. 7 shows dry weight (g/L) of cell pellets from cultures of Schizochytrium strain B76-32 transformed with pCL0076 and harvested after either 2 days or 7 days of growth in sucrose-SSFM. 2118* refers to a sub-isolate of wild-type Schizochytrium sp. ATCC 20888 cells grown on glucose-SSFM. B76-32** refers to the B76-32 parent strain grown on glucose-SSFM.

FIG. 8 shows fat content of cell pellets from cultures of Schizochytrium strain B76-32 transformed with pCL0076 and harvested after either 2 days or 7 days of growth in sucrose-SSFM. The rightmost column for each sample shows fat content as % weight of the dry biomass. The leftmost column for each sample shows % of total fat composed of acyl groups with 18 or fewer carbons (light grey) or 20 or more carbons (medium grey). 2118* refers to a sub-isolate of wild-type Schizochytrium sp. ATCC 20888 cells grown on glucose-SSFM. B76-32** refers to the B76-32 parent strain grown on glucose-SSFM.

FIG. 9A shows a Western blot for invertase protein and FIG. 9B shows a corresponding Coomassie-stained SDS-PAGE gel. S. cerevisiae invertase control and supernatants of a 3-day culture of pCL0076 transformant 1-3 were loaded in amounts of 5 μg, 2.5 μg, 1.25 μg, and 0.625 μg, respectively, as indicated at the top of the Western blot.

FIG. 10A shows an invertase activity assay illustrated by the reaction rate as a function of sucrose concentration. FIG. 10B shows a standard Lineweaver-Burk plot used to calculate the Kₘ and Vₘₐₓ.

FIG. 11 shows N-glycan structures detected on Schizochytrium secreted proteins as determined by NSI-Total Ion Mapping of permethylated N-glycans.

FIG. 12 shows a table of glycan species obtained by NSI-Total Ion Mapping of permethylated N-glycans.

FIG. 13 shows a plasmid map of pCL0120 (SEQ ID NO:5).

FIG. 14 shows a codon-optimized nucleic acid sequence (SEQ ID NO:4) encoding the Sec1 signal peptide from Schizochytrium fused to the mature Suc1 invertase from Aspergillus niger (GenBank Accession No. S33920).

FIG. 15 shows a plasmid map of pCL0137_EPCT(+)-s1Suc1, also termed pCL0137.

### DETAILED DESCRIPTION OF THE INVENTION

The present invention is directed to recombinant thraustochytrids that grow on sucrose and cell cultures comprising the recombinant thraustochytrids, as well as methods of producing cell cultures, biomasses, microbial oils, compositions, and biofuels using the recombinant thraustochytrids.

### Thraustochytrids

According to the present invention, the term “thraustochytrid” refers to any member of the order Thraustochytriales, which includes the family Thraustochytriaceae. The current taxonomic placement of the thraustochytrids can be summarized as follows:

```
Realm: Stramenopila (Chromista)
    Phylum: Labyrinthulomycota (Heterokonta)
        Class: Labyrinthulomycetes (Labyrinthulae)
            Order: Labyrinthulales
                Family: Labyrinthulaceae
            Order: Thraustochytriales
                Family: Thraustochytriaceae
```

For purposes of the present invention, strains described as thraustochytrids include the following organisms: Order: Thraustochytriales; Family: Thraustochytriaceae; Genera: Thraustochytrium (Species: sp., arudimentale, aureum, benthicola, globosum, kinnei, motivum, multirudimentale, pachydermum, proliferum, roseum, striatum), Ulkenia (Species: sp., amoeboidea, kerguelensis, minuta, profunda, radiata, sailens, sarkariana, schizochytrops, visurgensis, yorkensis), Schizochytrium (Species: sp., aggregatum, limnaceum, mangrovei, minutum, octosporum), Japonochytrium (Species: sp., marinum), Aplanochytrium (Species: sp., haliotidis, kerguelensis, profunda, stocchinoi), Althornia (Species: sp., crouchii), or Elina (Species: sp., marisalba, sinorifica). For the purposes of this invention, species described within Ulkenia will be considered to be members of the genus Thraustochytrium. Aurantiochytrium, Oblongichytrium, Botryochytrium, Parietichytrium, and Sicyoidochytrium are additional genuses encompassed by the present invention.

Strains of Thraustochytriales include, but are not limited to, deposited strains PTA-10212, PTA-10213, PTA-10214, PTA-10215, PTA-9695, PTA-9696, PTA-9697, PTA-9698, PTA-10208, PTA-10209, PTA-10210, PTA-10211, Thraustochytrium sp. (23B) (ATCC 20891); Thraustochytrium striatum (Schneider) (ATCC 24473); Thraustochytrium aureum (Goldstein) (ATCC 34304); Thraustochytrium roseum (Goldstein) (ATCC 28210); and Japonochytrium sp. (L1) (ATCC 28207). Schizochytrium include, but are not limited to Schizochytrium aggregatum, Schizochytrium limacinum, Schizochytrium sp. (S31) (ATCC 20888), Schizochytrium sp. (S8) (ATCC 20889), Schizochytrium sp. (LC-RM) (ATCC 18915), Schizochytrium sp. (SR 21), deposited strain ATCC 28209 and deposited Schizochytrium limacinum strain IFO 32693.

In some embodiments, the thraustochytrid cell of the invention is a Schizochytrium or a Thraustochytrium cell. In some embodiments, the thraustochytrid is from a species selected from Schizochytrium sp., Schizochytrium aggregatum, Schizochytrium limacinum, Schizochytrium minutum, Thraustochytrium sp., Thraustochytrium striatum, Thraustochytrium aureum, Thraustochytrium roseum, Japonochytrium sp., and strains derived therefrom.

According to the present invention, the term “transformation” is used to refer to any method by which a nucleic acid molecule (i.e., a recombinant nucleic acid molecule) can be inserted into a thraustochytrid cell of the invention. In microbial systems, the term “transformation” is used to describe an inherited change due to the acquisition of exogenous nucleic acids by the microorganism and is essentially synonymous with the term “transfection.” Suitable transformation techniques for introducing nucleic acid molecules into thraustochytrid cells include, but are not limited to, particle bombardment, electroporation, microinjection, lipofection, adsorption, infection, and protoplast fusion.

Although the phrase “nucleic acid molecule” primarily refers to the physical nucleic acid molecule and the phrases “nucleic acid sequence” or “polynucleotide sequence” primarily refers to the sequence of nucleotides in the nucleic acid molecule, the phrases are used interchangeably, especially with respect to a nucleic acid molecule, polynucleotide sequence, or a nucleic acid sequence that is capable of encoding a protein. In some embodiments, isolated nucleic acid molecules as described herein are produced using recombinant DNA technology (e.g., polymerase chain reaction (PCR) amplification or cloning) or chemical synthesis. In accordance with the present invention, an “isolated” nucleic acid molecule is a nucleic acid molecule that has been removed from its natural milieu (i.e., that has been subject to human manipulation), its natural milieu being the genome or chromosome in which the nucleic acid molecule is found in nature. As such, “isolated” does not necessarily reflect the extent to which the nucleic acid molecule has been purified, but indicates that the molecule does not include an entire genome or an entire chromosome in which the nucleic acid molecule is found in nature. An isolated nucleic acid molecule can include DNA, RNA (e.g., mRNA), or derivatives of either DNA or RNA (e.g., cDNA). Isolated nucleic acid molecules include natural nucleic acid molecules and homologues thereof, including, but not limited to, natural allelic variants and modified nucleic acid molecules in which nucleotides have been inserted, deleted, substituted, and/or inverted in such a manner that such modifications provide the desired effect on sequence, function, and/or the biological activity of the encoded peptide or protein.

The term “protein” includes single-chain polypeptide molecules as well as multiple-polypeptide complexes where individual constituent polypeptides are linked by covalent or non-covalent means. The term “polypeptide” includes peptides of two or more amino acids in length, typically having more than 5, 10, or 20 amino acids. In some embodiments, a polypeptide as described herein is a heterologous polypeptide. The term “heterologous” as used herein refers to a polypeptide (or polynucleotide) sequence, for example, that is not naturally found in the thraustochytrid cell of the invention.

Schizochytrium naturally uses hexose sugars and glycerol as a carbon source instead of sucrose. Examination of the genome database of Schizochytrium did not reveal genes required for the initial steps of sucrose catabolism, supporting the general finding that thraustochytrids metabolize glucose and/or fructose, which are the two sugar components of sucrose, but do not naturally metabolize sucrose. See also, e.g., Aki et al., JAOCS 80:789-794 (2003); Yokochi et al., Appl. Microbiol. Biotechnol. 49:72-76 (1998); Honda et al., Mycol. Res. 4:439-448 (1998); Singh, World J. of Microbiology 12:76-81 (1996); Goldstein, American J. of Botany 50:271-279 (1963); and U.S. Pat. No. 5,340,742.

The present invention is directed to a thraustochytrid cell transformed with a nucleic acid molecule comprising a polynucleotide sequence encoding a heterologous polypeptide associated with sucrose metabolism. In some embodiments, the polypeptide is a heterologous polypeptide. In some embodiments, a thraustochytrid cell of the invention comprises one or more nucleic acid molecules comprising one or more polynucleotide sequences encoding one or more heterologous sucrases, a heterologous sucrose transporter, or a combination thereof. In some embodiments, the thraustochytrid cell comprises a nucleic acid molecule comprising a polynucleotide sequence encoding a secreted sucrase. In some embodiments, the thraustochytrid cell comprises a nucleic acid molecule comprising a polynucleotide sequence encoding a cytoplasmic sucrase. In some embodiments, the thraustochytrid cell comprises a nucleic acid molecule comprising a polynucleotide sequence encoding a sucrose transporter. In some embodiments, the thraustochytrid cell comprises one or more nucleic acid molecules comprising one or more polynucleotides encoding one or more heterologous sucrases and a heterologous sucrose transporter. In some embodiments, the thraustochytrid cell comprises one or more nucleic acid molecules comprising one or more polynucleotides encoding a heterologous cytoplasmic invertase and a heterologous sucrose transporter. In some embodiments, the thraustochytrid cell comprises one or more nucleic acid molecules comprising one or more polynucleotides encoding a heterologous secreted invertase and a heterologous sucrose transporter. In some embodiments, the thraustochytrid cell comprises one or more nucleic acid molecules comprising one or more polynucleotides encoding a heterologous cytoplasmic invertase, a heterologous secreted invertase, and a heterologous sucrose transporter. In some embodiments, the polynucleotide sequence is codon-optimized to maximize translation efficiency in the thraustochytrid cell. In some embodiments, the polynucleotide sequence encoding the sucrase, sucrose transporter, or combination thereof is integrated into the genome of the thraustochytrid cell. In some embodiments, the polynucleotide sequence encoding the sucrase, sucrose transporter, or combination thereof is stably integrated into the genome of the thraustochytrid cell. Sucrases are part of a family of enzymes known as glycoside hydrolases (also termed glycosidases) that catalyze the hydrolysis of glycosidic linkages to generate two smaller sugars. As described herein, a “sucrase” is any enzyme that catalyzes the hydrolysis of sucrose (also known as saccharose or table sugar) to fructose and glucose. In certain embodiments, the sucrase is an invertase, a sucrase-isomaltase, a levanase, sacridase, beta-fructosidase, beta-fructofuranosidases, fructohydrolase, saccharase, inulinase, sucrose alpha-glucohydrolase, alpha-D-glucosidase, or a combination thereof. In some embodiments the sucrase is the invertase SUC2 from Saccharomyces cerevisiae; the invertase SacC from Zymomonas; the invertase INV1 from Pichia; the invertase INV from Debaryomyces; the sucrase Suc1 from Aspergillus; a sucrase from other fungal, protist, algal, plant, or eukaryotic sources; or any combinations thereof. In certain embodiments, the sucrase is from a prokaryotic source such as coryneform bacteria or Escherichia coli. In some embodiments, a sucrase expressed in a thraustochytrid cell of the invention comprises a different glycosylation pattern than when expressed in the organism from which the sucrase is derived. In some embodiments, the sucrase that is expressed in the thraustochytrid cell is a glycoprotein comprising high-mannose oligosaccharides. In some embodiments, the thraustochytrid cell also comprises a polynucleotide sequence encoding an enzyme required for saccharification of a cellulose feedstock (e.g., sugar cane). See, e.g., U.S. 2009/0064567, incorporated by reference herein in its entirety. In some embodiments, the thraustochytrid cell also comprises a polynucleotide sequence encoding a fructokinase, a glucokinase, a hexokinase, or a combination thereof.

In some embodiments, the expression system used for the production of a sucrase, a sucrose transporter, or a combination thereof in a thraustochytrid cell comprises regulatory elements that are derived from thraustochytrid sequences. In some embodiments, the expression system used for the production of a sucrase, a sucrose transporter, or a combination thereof in a thraustochytrid cell comprises regulatory elements that are derived from non-thraustochytrid sequences, including sequences derived from non-thraustochytrid algal sequences. In some embodiments, the expression system comprises a polynucleotide sequence encoding a sucrase, a sucrose transporter, or the combination of the two wherein the polynucleotide sequence is operably linked to any promoter sequence, any terminator sequence, and/or any other regulatory sequence that is functional in a thraustochytrid cell. In certain embodiments, the expression system comprises polynucleotide sequences encoding a cytoplasmically expressed sucrase and a sucrose transporter. Inducible or constitutively active regulatory sequences can be used. Regulatory sequences include but are not limited to the Schizochytrium regulatory sequences described in U.S. Publ. No. 2010/0233760, U.S. Pat. No. 7,001,772, and U.S. Publ. Nos. 2006/0275904 and 2006/0286650, incorporated by reference herein in their entireties, such as: an OrfC promoter, an OrfC terminator, an EF1 short promoter, EF1 long promoter, a Sec1 promoter, 60S short promoter, 60S long promoter, an acetolactate synthase promoter, an acetolactate synthase terminator, an α-tubulin promoter, a promoter from a polyketide synthase (PKS) system, a fatty acid desaturase promoter, an actin promoter, an actin terminator, an elongation factor 1 alpha (ef1α) promoter, an ef1α terminator, a glyceraldehyde 3-phosphate dehydrogenase (gapdh) promoter, a gapdh terminator, and combinations thereof.

In some embodiments, the sucrase is secreted into the fermentation medium by a recombinant thraustochytrid transformed with a sucrase gene. In some embodiments, the sucrase is a secreted protein comprising a signal peptide. In some embodiments, the signal peptide is naturally associated with the exogenous sucrase (i.e., the signal peptide is the native signal sequence of the exogenous sucrase). In some embodiments, the signal peptide is not naturally associated with the exogenous sucrase but is instead a heterologous signal peptide, such as a signal peptide from a microorganism of the phylum Labyrinthulomycota. In some embodiments, the signal peptide is from a thraustochytrid. In some embodiments, the signal peptide is from a Schizochytrium or a Thraustochytrium. In some embodiments, the signal peptide is the Schizochytrium Sec1 signal peptide (SEQ ID NO:3). A “signal peptide” includes full-length peptides and functional fragments thereof, fusion peptides, and homologues of a naturally occurring signal peptide. A homologue of a signal peptide differs from a naturally occurring signal peptide in that at least one or a few, but not limited to one or a few, amino acids have been deleted (e.g., a truncated version of the protein, such as a peptide or fragment), inserted, inverted, substituted, and/or derivatized (e.g., by glycosylation, phosphorylation, acetylation, acylation (e.g., myristoylation and palmitoylation), prenylation, amidation, and/or addition of glycosylphosphatidyl inositol). In some embodiments, homologues of a signal peptide retain activity as a signal at least in a thraustochytrid, although the activity can be increased, decreased, or made dependent upon certain stimuli. In some embodiments, the signal peptide sequence includes, but is not limited to, a Schizochytrium sequence described in U.S. Publ. No. 2010/0233760, such as: a Na/Pi transporter signal peptide, a shortened Na/Pi transporter signal peptide, an alpha-1,6-mannosyltransferase (ALG12) signal peptide, a binding immunoglobulin protein (BiP) signal peptide, an alpha-1,3-glucosidase (GLS2) signal peptide, an alpha-1,3-1,6-mannosidase-like signal peptide, an alpha-1,3-1,6-mannosidase-like #1 signal peptide, an alpha-1,2-mannosidase-like signal peptide, a beta-xylosdiase-like signal peptide, a carotene synthase signal peptide, and a Sec1 signal peptide.

In some embodiments, the sucrase is expressed cytoplasmically along with expression of a sucrose transporter. In some embodiments, the sucrase is membrane bound, e.g., sucrase-isomaltase, which has an N-terminal membrane spanning domain (signal anchor). In certain embodiments, the sucrase is localized to the exterior of the plasma membrane. In some embodiments, the sucrase is fused to a membrane spanning domain.

In some embodiments, an expression cassette is used for the expression of a sucrase, a sucrose transporter, or a combination thereof in a thraustochytrid cell. The design and construction of expression cassettes use standard molecular biology techniques known to persons skilled in the art. See, e.g., Sambrook et al., 2001, Molecular Cloning: A Laboratory Manual, 3^{rd} edition. In some embodiments, the thraustochytrid cell comprises an expression cassette containing genetic elements, such as at least the following operably linked in such a way that they are functional in the thraustochytrid cell: a promoter; a coding sequence comprising a sucrase, a sucrose transporter, or a combination thereof, and a terminator region. In some embodiments, the expression cassette further comprises a polynucleotide sequence encoding a signal peptide or a membrane domain operably linked to the sucrase or sucrose transporter. The term “membrane domain” as used herein refers to any domain within a polypeptide that targets the polypeptide to a membrane and/or allows the polypeptide to maintain association with a membrane and includes, but is not limited to, a transmembrane domain (e.g., a single or multiple membrane spanning region), an integral monotopic domain, a signal anchor sequence, an ER signal sequence, an N-terminal or internal or C-terminal stop transfer signal, a glycosylphosophatidylinositol anchor, and combinations thereof. A membrane domain can be located at any position in the polypeptide, including the N-terminal, C-terminal, or middle of the polypeptide. A membrane domain can be associated with permanent or temporary attachment of a polypeptide to a membrane. In some embodiments, a recombinant vector comprises the expression cassette. Recombinant vectors include, but are not limited to, plasmids, phages, and viruses. In some embodiments, the recombinant vector is a linearized vector. In some embodiments, the recombinant vector is an expression vector. As used herein, the phrase “expression vector” refers to a vector that is suitable for production of an encoded product (e.g., a sucrase, a sucrose transporter, or a combination thereof). In some embodiments, a polynucleotide sequence encoding the sucrase, sucrose transporter, or combination thereof is inserted into the recombinant vector to produce a recombinant nucleic acid molecule. In some embodiments, the polynucleotide sequence encoding the sucrase, sucrose transporter, or combination thereof is inserted into the vector in a manner that operatively links the nucleic acid sequence to regulatory sequences in the vector, which enables the transcription and translation of the nucleic acid sequence within the recombinant microorganism. In some embodiments, a selectable marker enables the selection of a recombinant microorganism into which a recombinant nucleic acid molecule of the present invention has successfully been introduced. Selectable markers include but are not limited to the selectable markers described in U.S. Publ. No. 2010/0233760 and U.S. Pat. No. 7,001,772, such as Schizochytrium acetolactate synthase or bacterial ble. In some embodiments, the selection marker is an auxotrophic marker, a dominant selection marker (such as, for example, an enzyme that degrades antibiotic activity), or another protein involved in transformation selection.

In some embodiments, the thraustochytrid cell is genetically modified to introduce or delete genes involved in biosynthetic pathways associated with the transport and/or synthesis of carbohydrates, including those involved in glycosylation. For example, the thraustochytrid cell can be modified by deleting endogenous glycosylation genes and/or inserting human or animal glycosylation genes to allow for glycosylation patterns that more closely resemble those of humans. Modification of glycosylation in yeast is exemplified in, for example, U.S. Pat. No. 7,029,872 and U.S. Publ. Nos. 2004/0171826, 2004/0230042, 2006/0257399, 2006/0029604, and 2006/0040353. In some embodiments, the thraustochytrid cell includes a cell in which RNA viral elements are employed to increase or regulate gene expression.

In some embodiments, a polynucleotide sequence of any of the methods of the invention encodes a heterologous polypeptide comprising an amino acid sequence that is at least 90%, at least 91%, at least 92%, at least 93%, at least 94%, at least 95%, at least 96%, at least 97%, at least 98%, at least 99%, or that is identical to the amino acid sequence of a polypeptide associated with sucrose metabolism including, but not limited to, a polypeptide associated with a sucrase (such as, for example, an invertase, sucrase-isomaltase, levanase, sacridase, beta-fructosidase, beta-fructofuranosidases, fructohydrolase, saccharase, inulinase, sucrose alpha-glucohydrolase, alpha-D-glucosidase) or a sucrose transporter. In some embodiments, a polynucleotide sequence of any of the methods of the invention encodes a heterologous polypeptide comprising an amino acid sequence that is at least 90%, at least 91%, at least 92%, at least 93%, at least 94%, at least 95%, at least 96%, at least 97%, at least 98%, at least 99%, or that is identical to: the amino acid sequence of the invertase SUC2 from Saccharomyces cerevisiae; the invertase SacC from Zymomonas; the invertase INV1 from Pichia; the invertase INV from Debaryomyces; the sucrase Suc1 from Aspergillus; a sucrase from other fungal, protist, algal, plant, or eukaryotic sources; or a sucrase from a prokaryotic source such as coryneform bacteria or Escherichia coli. In some embodiments, a polynucleotide sequence of any of the methods of the invention encodes a polypeptide comprising an amino acid sequence that is at least 90%, at least 91%, at least 92%, at least 93%, at least 94%, at least 95%, at least 96%, at least 97%, at least 98%, at least 99%, or that is identical to: the amino acid sequence encoded by Accession No. L47346 or X69165; or the amino acid sequence of Accession No. P00724 or S33920. In some embodiments, a polynucleotide sequence of any of the methods of the invention encodes a polypeptide comprising an amino acid sequence that is at least 90%, at least 91%, at least 92%, at least 93%, at least 94%, at least 95%, at least 96%, at least 97%, at least 98%, at least 99%, or that is identical to the amino acid sequence encoded by SEQ ID NO:1 or SEQ ID NO:4. In some embodiments, a polynucleotide sequence of any of the methods of the invention is at least 90%, at least 91%, at least 92%, at least 93%, at least 94%, at least 95%, at least 96%, at least 97%, at least 98%, at least 99%, or that is identical to: the polynucleotide sequence of Accession No. L47346 or X69165; a polynucleotide sequence of Accession No. L47346 or X69165 that is codon-optimized for expression in Schizochytrium; a polynucleotide sequence encoding the amino acid sequence of Accession No. P00724 or S33920; a polynucleotide sequence encoding the amino acid sequence of Accession No. P00724 or S33920, wherein the polynucleotide sequence is codon-optimized for expression in Schizochytrium; or the polynucleotide sequence of SEQ ID NO:1 or SEQ ID NO:4.

### Thraustochytrid Cultures, Biomasses, and Microbial Oils

The present invention is also directed to a thraustochytrid cell culture comprising any of the thraustochytrid cells of the invention as described herein and a cell culture medium comprising sucrose as a carbon source.

The present invention is also directed to a method of producing a thraustochytrid cell culture, comprising transforming a thraustochytrid cell with a nucleic acid molecule comprising a polynucleotide sequence encoding a polypeptide associated with sucrose metabolism as described herein, selecting the transformed thraustochytrid cell, and growing the transformed thraustochytrid cell in a culture medium comprising sucrose as a carbon source, wherein a thraustochytrid cell culture is produced. The present invention is also directed to a thraustochytrid cell culture produced by the method.

In some embodiments, sucrose is the primary carbon source in the cell culture medium. In some embodiments, sucrose is the only carbon source in the cell culture medium. Sucrose sources include but are not limited to sugar cane, sugar beets, and other plants. The composition of the purified or partially purified sucrose can be predominantly sucrose, i.e., purified/refined sucrose, crude sucrose extracts, or a mixture of sucrose, glucose, and fructose (e.g., molasses).

In some embodiments, the culture medium comprises molasses, a syrup, or juice from any sucrose-producing vegetable (e.g., sugar cane juice or sugar beet juice), or combinations thereof. In some embodiments, the culture medium comprises a cellulose-containing feedstock, such as sugar cane bagasse. In some embodiments, the cell culture medium comprises a sucrase. In some embodiments, the thraustochytrid cell secretes a heterologous sucrase into the culture medium.

Culture conditions for thraustochytrid cells include, but are not limited to, effective media, bioreactor, temperature, pH, and oxygen conditions that permit protein production and/or recombination. An effective medium refers to any medium in which a thraustochytrid cell is typically cultured. Such medium typically comprises an aqueous medium having assimilable carbon, nitrogen, and phosphate sources, as well as appropriate salts, minerals, metals, and other nutrients, such as vitamins. Nitrogen sources include, but are not limited to, peptone, yeast extract, polypeptone, malt extract, meat extract, casamino acid, corn steep liquor, organic nitrogen sources, sodium glutamate, urea, inorganic nitrogen sources, ammonium acetate, ammonium sulfate, ammonium chloride, ammonium nitrate, and sodium sulfate. Non-limiting culture conditions suitable for thraustochytrids are described, for example, in U.S. Pat. No. 5,340,742. Various fermentation parameters for inoculating, growing, and recovering microflora are also described, for example, in U.S. Pat. No. 5,130,242. Liquid or solid media can contain natural or artificial sea water. Thraustochytrid cells of the present invention can be cultured in fermentation bioreactors, shake flasks, test tubes, microtiter dishes, and petri plates.

The fermentation volume can be any volume used for the growth of thraustochytrids, including commercial and industrial volumes. In some embodiments, the fermentation volume (volume of culture) is at least 2 L, at least 10 L, at least 50 L, at least 100 L, at least 200 L, at least 500 L, at least 1000 L, at least 10,000 L, at least 20,000 L, at least 50,000 L, at least 100,000 L, at least 150,000 L, at least 200,000 L, or at least 250,000 L, at least 300,000 L, at least 350,000 L, at least 400,000 L, or at least 500,000 L. In some embodiments, the fermentation volume is 2 L to 2,000,000 L, 2 L to 1,000,000 L, 2 L to 500,000 L, 2 L to 200,000 L, 2 L to 100,000 L, 2 L to 50,000 L, 2 L to 25,000 L, 2 L to 20,000 L, 2 L to 15,000 L, 2 L to 10,000 L, 2 L to 5,000 L, 2 L to 1,000 L, 2 L to 500 L, or 2 L to 100 L.

The present invention is also directed to a method of producing a thraustochytrid biomass comprising growing a thraustochytrid cell of the invention in a culture medium comprising sucrose as a carbon source, and harvesting a biomass from the culture medium. A thraustochytrid biomass is a harvested cellular biomass obtained by any conventional method for the isolation of a thraustochytrid biomass, such as described in U.S. Pat. No. 5,130,242 and U.S. Publ. No. 2002/0001833, incorporated by reference herein in their entireties. A harvested biomass can contain thraustochytrid cells as well as thraustochytrid cellular fragments.

The present invention is also directed to a method of producing a microbial oil comprising growing a thraustochytrid cell of the invention in a culture medium comprising sucrose as a carbon source to produce a biomass, and extracting an oil from the biomass. The oil can be extracted from a freshly harvested biomass or can be extracted from a previously harvested biomass that has been stored under conditions that prevent spoilage. Known methods can be used to culture a thraustochytrid of the invention, to isolate a biomass from the culture, to extract a microbial oil from the biomass, and to analyze the fatty acid profile of oils extracted from the biomass. See, e.g., U.S. Pat. No. 5,130,242.

A microbial oil can be any oil derived from any thraustochytrid, including, for example: a crude oil extracted from the biomass of a thraustochytrid without further processing; a refined oil that is obtained by treating a crude oil with further processing such as refining, bleaching, and/or deodorizing; a diluted oil obtained by diluting a crude or refined oil; or an enriched oil that is obtained, for example, by treating a crude or refined oil with further methods of purification to increase the concentration of a fatty acid in the oil.

In some embodiments, the crude oil can be isolated from a thraustochytrid using standard techniques, without being subjected to further refinement or purification. For example, the crude oil can be isolated using solvent extraction, such as, but not limited to, hexane extraction or isopropanol extraction. In some embodiments, the crude oil can be isolated using physical and/or mechanical extraction methods such as, but not limited to, extraction through the use of a homogenizer or by pressing.

In some embodiments, the crude oil can be subjected to further processing, such as refining, desolventization, deodorization, winterization, chill filtration, and/or bleaching. Such “processed” oils include microbial oils that have been subjected to solvent extraction and one or more further processing. In some embodiments, oils are minimally processed. “Minimally processed” oils include microbial oils that have been subjected to solvent extraction and filtration. In certain embodiments, minimally processed oils are further subjected to winterization.

In some embodiments, a method similar to the FRIOLEX® process (Westfalia Separator Industry GmbH, Germany) is used to extract the microbial oils produced by the thraustochytrids. The FRIOLEX® process is a water-based physical oil extraction process, whereby raw material containing oil can be used directly for extracting oil without using any conventional solvent extraction methods. In this process, a water-soluble organic solvent can be used as a process aid and the oil is separated from the raw material broth by density separation using gravity or centrifugal forces. WO 96/05278 discloses such extraction methods and is herein incorporated by reference in its entirety.

After the oil has been extracted, the oil can be recovered or separated from non-lipid components by any suitable means known in the art. In some embodiments, low-cost physical and/or mechanical techniques are used to separate the lipid-containing compositions from non-lipid compositions. For example, if multiple phases or fractions are created by the extraction method used to extract the oil, where one or more phases or fractions contain lipids, a method for recovering the lipid-containing phases or fractions can involve physically removing the lipid-containing phases or fractions from the non-lipid phases or fractions, or vice versa. In some embodiments, a FRIOLEX® type method is used to extract the lipids produced by the microorganisms, and the lipid-rich light phase is then physically separated from the protein-rich heavy phase (such as by skimming off the lipid-rich phase that is on top of the protein-rich heavy phase after density separation).

The microbial oils produced by thraustochytrids of the present invention can be recovered from autolysis or induced lysis by exposing thraustochytrid cells to a condition including, but not limited to, a certain pH, a certain temperature, the presence of an enzyme, the presence of a detergent, physical disruptions, or combinations thereof. In some embodiments, lysis or autolysis of the thraustochytrid cells is performed by the use of mechanical forces. In further embodiments of the present invention, the lysis or autolysis of thraustochytrid cells is followed by mechanical separation of the lipids from the non-lipid compositions.

Suitable enzymes that can be used to induce lysis of the thraustochytrid cells include, but are not limited to, commercially available enzymes or enzyme mixtures such as Proteinase K or ALCALASE®. In some embodiments, the oil-producing thraustochytrids undergo induced lysis in the presence of a detergent such as ionic (cationic or anionic) detergents, nonionic detergents, zwitterionic detergents, or combinations thereof. In further embodiments of the present invention, physical disruption methods such as mechanical grinding, liquid homogenization, use of high frequency sound waves in sonication, freeze/thaw cycles methods, pressing, extruding, or milling can be used to induce lysis of the oil-producing thraustochytrids. The extraction of the oils can take place in the fermentors at the end of the fermentation by in-tank lysis of the thraustochytrid cells.

In some embodiments, the cell cultures, biomasses, and microbial oils produced from the thraustochytrid cells of the invention grown on sucrose have the same or substantially similar characteristics (e.g., cell densities, dry cell weights, fatty acid productivities, and fatty acid profiles) associated with cultures, biomasses, and microbial oils produced from the corresponding untransformed thraustochytrid cells grown on glucose and/or fructose. In some embodiments, the dry cell weight of a biomass produced from a thraustochytrid culture of the invention is higher after growth on sucrose than the dry cell weight obtained under identical conditions from growth of a culture of the corresponding untransformed thraustochytrid cells on glucose and/or fructose. In some embodiments, the total amount of fatty acids as a percentage of the dry cell weight of the biomass is higher after growth of a thraustochytrid of the invention on sucrose than the amount obtained under identical conditions from growth of a corresponding untransformed thraustochytrid cell on glucose and/or fructose.

### Methods of Producing Compositions

The present invention is also directed to a method of preparing a food product, cosmetic, industrial composition, or pharmaceutical composition for animals or humans, comprising growing a thraustochytrid cell of the invention in a culture medium comprising sucrose as a carbon source to produce a biomass, harvesting the biomass, and preparing the food product, cosmetic, industrial composition, or pharmaceutical composition for animals or humans from the biomass.

Thraustochytrid biomasses can be dried prior to use in a composition by methods including, but not limited to, freeze drying, air drying, spray drying, tunnel drying, vacuum drying (lyophilization), or a similar process. Alternatively, a harvested and washed biomass can be used directly in a composition without drying. See, e.g., U.S. Pat. Nos. 5,130,242 and 6,812,009, incorporated by reference herein in their entireties.

In some embodiments, the method of preparing a food product, cosmetic, industrial composition, or pharmaceutical composition for animals or humans, further comprises extracting an oil from the biomass. Microbial oils can be used as starting material to more efficiently produce a product enriched in a fatty acid such as DHA or EPA. For example, the microbial oils can be subjected to various purification techniques known in the art, such as distillation or urea adduction, to produce a higher potency product with higher concentrations of DHA, EPA, or another fatty acid. The microbial oils can also be used in chemical reactions to produce compounds derived from fatty acids in the oils, such as esters and salts of DHA, EPA, or another fatty acid.

Any thraustochytrid that has been transformed as described herein to grow on sucrose can be selected for preparing any of the described compositions based on a desirable attribute of the thraustochytrid, such as but not limited to the cell density of a cell culture of the thraustochytrid, the percentages and types of fatty acids associated with the dry cell of a biomass of the thraustochytrid, the fatty acid profile associated with the biomass and/or the microbial oil of the thraustochytrid, or a combination thereof. In some embodiments, the thraustochytrid biomass or microbial oil comprises a greater percentage of polyunsaturated fatty acids than monounsaturated fatty acids. In some embodiments, the thraustochytrid biomass or microbial oil comprises a greater percentage of omega-3 fatty acids than omega-6 fatty acids. In some embodiments, the thraustochytrid biomass or microbial oil comprises a greater percentage of DHA than other omega-3 fatty acids. In some embodiments, the thraustochytrid biomass comprises a greater percentage of DHA than other polyunsaturated fatty acids. In some embodiments, the thraustochytrid biomass or microbial oil comprises a greater percentage of EPA than other omega-3 fatty acids. In some embodiments, the thraustochytrid biomass or microbial oil comprises a greater percentage of EPA than other polyunsaturated fatty acids.

A composition can include one or more excipients. As used herein, “excipient” refers to a component, or mixture of components, that is used in a composition to give desirable characteristics to the composition, including foods as well as pharmaceutical, cosmetic, and industrial compositions. An excipient can be described as a “pharmaceutically acceptable” excipient when added to a pharmaceutical composition, meaning that the excipient is a compound, material, composition, salt, and/or dosage form which is, within the scope of sound medical judgment, suitable for contact with the tissues of human beings and animals without excessive toxicity, irritation, allergic response, or other problematic complications over the desired duration of contact commensurate with a reasonable benefit/risk ratio. In some embodiments, the term “pharmaceutically acceptable” means approved by a regulatory agency of the Federal or a state government or listed in the U.S. Pharmacopeia or other generally recognized international pharmacopeia for use in animals, and more particularly in humans. Various excipients can be used. In some embodiments, the excipient can be, but is not limited to, an alkaline agent, a stabilizer, an antioxidant, an adhesion agent, a separating agent, a coating agent, an exterior phase component, a controlled-release component, a solvent, a surfactant, a humectant, a buffering agent, a filler, an emollient, or combinations thereof. Excipients in addition to those discussed herein can include excipients listed in, though not limited to, Remington: The Science and Practice of Pharmacy, 21^{st} ed. (2005).

In some embodiments, the composition is a food product. A food product is any food for animal or human consumption, and includes both solid and liquid compositions. A food product can be an additive to animal or human foods. Foods include, but are not limited to, common foods; liquid products, including milks, beverages, therapeutic drinks, and nutritional drinks; functional foods; supplements; nutraceuticals; infant formulas, including formulas for pre-mature infants; foods for pregnant or nursing women; foods for adults; geriatric foods; and animal foods.

In some embodiments, a thraustochytrid biomass or microbial oil can be used directly as or included as an additive within one or more of: an oil, shortening, spread, other fatty ingredient, beverage, sauce, dairy-based or soy-based food (such as milk, yogurt, cheese and ice-cream), a baked good, a nutritional product, e.g., as a nutritional supplement (in capsule or tablet form), a vitamin supplement, a diet supplement, a powdered drink, a finished or semi-finished powdered food product, and combinations thereof.

A partial list of food compositions that can include a microbial oil includes, but is not limited to, soya based products (milks, ice creams, yogurts, drinks, creams, spreads, whiteners); soups and soup mixes; doughs, batters, and baked food items including, for example, fine bakery wares, breakfast cereals, cakes, cheesecakes, pies, cupcakes, cookies, bars, breads, rolls, biscuits, muffins, pastries, scones, croutons, crackers, sweet goods, snack cakes, pies, granola/snack bars, and toaster pastries; candy; hard confectionery; chocolate and other confectionery; chewing gum; liquid food products, for example milks, energy drinks, infant formula, carbonated drinks, teas, liquid meals, fruit juices, fruit-based drinks, vegetable-based drinks; multivitamin syrups, meal replacers, medicinal foods, and syrups; powdered beverage mixes; pasta; processed fish products; processed meat products; processed poultry products; gravies and sauces; condiments (ketchup, mayonnaise, etc.); vegetable oil-based spreads; dairy products; yogurt; butters; frozen dairy products; ice creams; frozen desserts; frozen yogurts; semi-solid food products such as baby food; puddings and gelatin desserts; processed and unprocessed cheese; pancake mixes; food bars including energy bars; waffle mixes; salad dressings; replacement egg mixes; nut and nut-based spreads; salted snacks such as potato chips and other chips or crisps, corn chips, tortilla chips, extruded snacks, popcorn, pretzels, potato crisps, and nuts; specialty snacks such as dips, dried fruit snacks, meat snacks, pork rinds, health food bars and rice/corn cakes.

In some embodiments, a microbial oil can be used to supplement infant formula. Infant formula can be supplemented with a microbial oil alone or in combination with a physically refined oil derived from an arachidonic acid (ARA)-producing microorganism. An ARA-producing microorganism, for example, is Mortierella alpina or Mortierella sect. schmuckeri. Alternatively, infant formulas can be supplemented with a microbial oil in combination with an oil rich in ARA, including ARASCO® (Martek Biosciences, Columbia, Md.).

In some embodiments, the composition is an animal feed. An “animal” means any non-human organism belonging to the kingdom Animalia, and includes, without limitation, aquatic animals and terrestrial animals. The term “animal feed” or “animal food” refers to any food intended for non-human animals, whether for fish; commercial fish; ornamental fish; fish larvae; bivalves; mollusks; crustaceans; shellfish; shrimp; larval shrimp; artemia; rotifers; brine shrimp; filter feeders; amphibians; reptiles; mammals; domestic animals; farm animals; zoo animals; sport animals; breeding stock; racing animals; show animals; heirloom animals; rare or endangered animals; companion animals; pet animals such as dogs, cats, guinea pigs, rabbits, rats, mice, or horses; primates such as monkeys (e.g., cebus, rhesus, African green, patas, cynomolgus, and cercopithecus), apes, orangutans, baboons, gibbons, and chimpanzees; canids such as dogs and wolves; felids such as cats, lions, and tigers; equids such as horses, donkeys, and zebras; food animals such as cows, cattle, pigs, and sheep; ungulates such as deer and giraffes; rodents such as mice, rats, hamsters and guinea pigs; and so on. An animal feed includes, but is not limited to, an aquaculture feed, a domestic animal feed including pet feed, a zoological animal feed, a work animal feed, a livestock feed, or a combination thereof.

In some embodiments, the composition is a feed or feed supplement for any animal whose meat or products are consumed by humans, such as any animal from which meat, eggs, or milk is derived for human consumption. When fed to such animals, nutrients such as LC-PUFAs can be incorporated into the flesh, milk, eggs or other products of such animals to increase their content of these nutrients.

In some embodiments, the composition is a spray-dried material that can be crumbled to form particles of an appropriate size for consumption by zooplankton, artemia, rotifers, and filter feeders. In some embodiments, the zooplankton, artemia, or rotifers fed by the composition are in turn fed to fish larvae, fish, shellfish, bivalves, or crustaceans.

In some embodiments, the composition is a pharmaceutical composition. Suitable pharmaceutical compositions include, but are not limited to, an anti-inflammatory composition, a drug for treatment of coronary heart disease, a drug for treatment of arteriosclerosis, a chemotherapeutic agent, an active excipient, an osteoporosis drug, an anti-depressant, an anti-convulsant, an anti-Helicobacter pylori drug, a drug for treatment of neurodegenerative disease, a drug for treatment of degenerative liver disease, an antibiotic, a cholesterol lowering composition, and a triglyceride lowering composition. In some embodiments, the composition is a medical food. A medical food includes a food that is in a composition to be consumed or administered externally under the supervision of a physician and that is intended for the specific dietary management of a condition, for which distinctive nutritional requirements, based on recognized scientific principles, are established by medical evaluation.

In some embodiments, the microbial oil can be formulated in a dosage form. Dosage forms can include, but are not limited to, tablets, capsules, cachets, pellets, pills, powders and granules, and parenteral dosage forms, which include, but are not limited to, solutions, suspensions, emulsions, and dry powders comprising an effective amount of the microbial oil. It is also known in the art that such formulations can also contain pharmaceutically acceptable diluents, fillers, disintegrants, binders, lubricants, surfactants, hydrophobic vehicles, water soluble vehicles, emulsifiers, buffers, humectants, moisturizers, solubilizers, preservatives, and the like. Administration forms can include, but are not limited to, tablets, dragees, capsules, caplets, and pills, which contain the microbial oil and one or more suitable pharmaceutically acceptable carriers.

For oral administration, the microbial oil can be combined with pharmaceutically acceptable carriers well known in the art. Such carriers enable the microbial oils to be formulated as tablets, pills, dragees, capsules, liquids, gels, syrups, slurries, suspensions, and the like, for oral ingestion by a subject to be treated. In some embodiments, the dosage form is a tablet, pill or caplet. Pharmaceutical preparations for oral use can be obtained by adding a solid excipient, optionally grinding the resulting mixture, and processing the mixture of granules, after adding suitable auxiliaries, if desired, to obtain tablets or dragee cores. Suitable excipients include, but are not limited to, fillers such as sugars, including, but not limited to, lactose, sucrose, mannitol, and sorbitol; cellulose preparations such as, but not limited to, maize starch, wheat starch, rice starch, potato starch, gelatin, gum tragacanth, methyl cellulose, hydroxypropylmethyl cellulose, sodium carboxymethyl cellulose, and polyvinylpyrrolidone (PVP). If desired, disintegrating agents can be added, such as, but not limited to, the cross-linked polyvinyl pyrrolidone, agar, or alginic acid or a salt thereof such as sodium alginate. Pharmaceutical preparations that can be used orally include, but are not limited to, push-fit capsules made of gelatin, as well as soft, sealed capsules made of gelatin and a plasticizer, such as glycerol or sorbitol.

In some embodiments, the composition is a cosmetic. Cosmetics include, but are not limited to, emulsions, creams, lotions, masks, soaps, shampoos, washes, facial creams, conditioners, make-ups, bath agents, and dispersion liquids. Cosmetic agents can be medicinal or non-medicinal.

In some embodiments, the composition is an industrial composition. In some embodiments, the composition is a starting material for one or more manufactures. A manufacture includes, but is not limited to, a polymer; a photographic photosensitive material; a detergent; an industrial oil; or an industrial detergent. For example, U.S. Pat. No. 7,259,006 describes use of DHA-containing fat and oil for production of behenic acid and production of photographic sensitive materials using behenic acid.

### Methods of Producing Biofuels

The present invention is also directed to a method for producing a biofuel from a recombinant thraustochytrid of the invention.

As used herein, “biofuel” refers to any fuel that is produced from or using a biomass or a microbial oil of a thraustochytrid of the invention, including, but not limited to, a biodiesel, a renewable diesel, a co-processed renewable diesel, a jet biofuel, a heating oil, and a fuel additive. Any method known in the art can be used to generate a biofuel from the biomasses and microbial oils of the thraustochytrids of the present invention, including but not limited to any methods described in WO 2005/035693, U.S. Publ. No. 2005/0112735, 2007/027633, WO 2006/127512, U.S. Publ. No. 2007/0099278, U.S. Publ. No. 2007/0089356, WO 2008/067605, U.S. Publ. No. 2009/0035842, and U.S. Publ. No. 2009/0064567, incorporated by reference herein in their entireties. Any of the biofuels described herein can be blended with a fossil fuel. For example, a biodiesel can be blended with a fossil diesel at ratios of 1%-99%. In some embodiments, a microbial oil of a thraustochytrid of the invention is used as a starting point for production of a biofuel even though it has not been subjected to conventional processing. Examples of conventional processing that can be avoided include refining (e.g., physical refining, silica refining or caustic refining), desolventization, deodorization, winterization, chill filtration, and/or bleaching. Thus, in certain embodiments, the oils have not been subjected to refining, desolventization, deodorization, winterization, chill filtration, bleaching, or a combination thereof.

In some embodiments, the method of producing a biofuel comprises growing a thraustochytrid of the invention in a culture medium comprising sucrose as a carbon source to produce a biomass, extracting an oil from the biomass, and producing a biofuel by transesterifying the oil, cracking the oil, processing the oil by thermal depolymerization, adding the oil to a traditional petroleum refining process, or a combination thereof.

In some embodiments, the biofuel is produced by transesterifying the oil. In some embodiments, the biofuel is a biodiesel. Various forms of biodiesel are known and include, but are not limited to, biodiesels described in WO 07/061,903, U.S. Pat. No. 7,172,635, EP Pat. No. 1 227 143, WO 02/38709, WO 02/38707, and U.S. Publ. No. 2007/0113467. Transesterification of triglycerides in the oil produces long chain fatty acid esters, i.e., alkyl esters, and can be performed by any method known in the art for the production of biofuels. In some embodiments, the alkyl ether is a methyl ester or an ethyl ester. In some embodiments, the extracting the oil from the thraustochytrid biomass and transesterifying the oil can be performed simultaneously. In some embodiments, the extracting the oil from the thraustochytrid biomass and transesterifying the oil are performed separately. See, e.g., U. S. Publ. No. 2009/0064567.

In some embodiments, transesterification is performed using a lower alkyl alcohol and an acid or base catalyst. Alcohols suitable for use in the present invention include any lower alkyl alcohol containing from 1 to 6 carbon atoms (i.e., a C₁₋₆ alkyl alcohol, such as methyl, ethyl, isopropyl, butyl, pentyl, hexyl alcohols and isomers thereof). In some embodiments, the alcohol comprises from 5 wt. % to 70 wt. %, 5 wt. % to 60 wt. %, 5 wt. % to 50 wt. %, 7 wt. % to 40 wt. %, 9 wt. % to 30 wt. %, or 10 wt. % to 25 wt. % of the mixture of the oil composition, the alcohol, and a catalytic base. In some embodiments, the oil composition and the base can be added to either pure ethanol or pure methanol. The amount of alcohol used can vary with the solubility of the oil in the alcohol. Any base known in the art to be suitable for use as a reactant can be used, including bases of the formula RO-M, wherein M is a monovalent cation and RO is an alkoxide of a C₁₋₆ alkyl alcohol. Examples of suitable bases include, but are not limited to, elemental sodium, sodium methoxide, sodium ethoxide, potassium methoxide, and potassium ethoxide. In some embodiments, the base is sodium ethoxide. In some embodiments, the base is added to the reaction with the oil composition and the alcohol in an amount of from 0.05 to 2.0 molar equivalents of triglycerides in the oil, 0.05 to 1.5 molar equivalents, 0.1 to 1.4 molar equivalents, 0.2 to 1.3 molar equivalents, or 0.25 to 1.2 molar equivalents. The oil comprising triglycerides, the alcohol, and the base are reacted together at a temperature and for an amount of time that allows the production of an ester from the fatty acid residues and the alcohol. In some embodiments, the reacting of the composition in the presence of an alcohol and a base is performed at a temperature from 20° C. to 140° C., from 20° C. to 120° C., from 20° C. to 110° C., from 20° C. to 100° C., or from 20° C. to 90° C. In some embodiments, the reacting of the composition in the presence of an alcohol and a base is performed at a temperature of at least 20° C., at least 75° C., at least 80° C., at least 85° C., at least 90° C., at least 95° C., at least 105° C., or at least 120° C. In some embodiments, the reacting of the composition in the presence of an alcohol and a base is performed at a temperature of 20° C., 75° C., 80° C., 85° C., 90° C., 95° C., 105° C., or 120° C. In some embodiments, the reacting of the composition in the presence of an alcohol and a base is performed for a time from 2 hours to 36 hours, from 3 hours to 36 hours, from 4 hours to 36 hours, from 5 hours to 36 hours, or from 6 hours to 36 hours. In some embodiments, the reacting of the composition in the presence of an alcohol and a base is performed for 0.25, 0.5, 1, 2, 4, 5, 5.5, 6, 6.5, 7, 7.5, 8, 8.5, 10, 12, 16, 20, 24, 28, 32, or 36 hours. In some embodiments, the reacting of the oil composition, alcohol, and base can be conducted by refluxing the components to produce the fatty acid esters, such as PUFA esters. In some embodiments, the reacting of the oil composition can be carried out at a temperature that does not result in the refluxing of the reaction components. For example, carrying out the reacting the oil composition under pressures greater than atmospheric pressure can increase the boiling point of the solvents present in the reaction mixture. Under such conditions, the reaction can occur at a temperature at which the solvents would boil at atmospheric pressure, but would not result in the refluxing of the reaction components. In some embodiments, the reaction is conducted at a pressure from 5 pounds per square inch (psi) to 20 psi; from 7 psi to 15 psi; or from 9 psi to 12 psi. In some embodiments, the reaction is conducted at a pressure of 7, 8, 9, 10, 11, or 12 psi. Reactions conducted under pressure can be carried out at the reaction temperatures listed above. In some embodiments, reactions conducted under pressure can be carried out at least from 20° C. to 140° C., from 20° C. to 120° C., from 20° C. to 110° C., from 20° C. to 100° C., or from 20° C. to 90° C. In some embodiments, reactions conducted under pressure can be carried out at least 70° C., at least 75° C., at least 80° C., at least 85° C., or at least 90° C. In some embodiments, reactions conducted under pressure can be carried out at 70° C., 75° C., 80° C., 85° C., or 90° C.

Reduced amounts of PUFA in an oil used for producing a biofuel, such as a biodiesel, can increase the energy density of the biofuel and can reduce the number of sites for potential oxidation or sulfation. In some embodiments, the thraustochytrid biomass or microbial oil, or triglyceride fraction thereof, comprises a greater percentage of monounsaturated fatty acids than polyunsaturated fatty acids. In some embodiments, the microbial oil is fractionated to remove polyunsaturated fatty acids. In some embodiments, the oil is hydrogenated in order to convert polyunsaturated fatty acids to monounsaturated fatty acids. To ensure that greater percentages of microalgal oil-derived biodiesel can be burned, any method of partial or total oil hydrogenation, as is routine in the manufacture of margarines, can be used. In some embodiments, thraustochytrids that produce low levels of PUFAs are used to produce the microbial oils. In some embodiments, thraustochytrids of the invention are selected that produce greater amounts of monounsaturated fatty acids than polyunsaturated fatty acids. In some embodiments, less than 50% of unsaturated fatty acids in the biological oil are PUFAs. In some embodiments, the unsaturated fatty acids in the biological oil contain less than 40%, less than 30%, less than 20%, less than 10%, or less than 5% PUFAs. In some embodiments, the microbial oil comprises less than 50%, less than 40%, less than 30%, less than 20%, less than 10%, or less than 5% by weight of PUFAs.

In addition to the transesterification methods described above, other techniques of reducing the viscosity of the microbial oils can also be used to produce biofuels. These techniques include, but are not limited to, the use of lipases, supercritical methanol catalysis, and the use of whole-cell systems involving cytoplasmic overexpression of lipases in a host cell followed by permeabilization of the host to allow catalysis of transesterification of triglycerides within the cytoplasm. In some embodiments, a thraustochytrid cell of the invention also comprises a polynucleotide sequence encoding a lipase for catalysis of transesterification of triglycerides within the cytoplasm. See, for example, U.S. Pat. No. 7,226,771, U.S. Publ. No. 2004/0005604, WO 03/089620, WO 05/086900, U.S. Publ. No. 2005/0108789, WO 05/032496, WO 05/108533, U.S. Pat. No. 6,982,155, WO 06/009676, WO 06/133698, WO 06/037334, WO 07/076,163, WO 07/056,786, and WO 06/124818, herein incorporated by reference in their entireties.

In some embodiments, the biofuel is produced by cracking the oil. In some embodiments, the biofuel is a jet biofuel. “Cracking” is understood in the art to describe the reduction of the chain length of fatty acids in an oil by methods such as those used in the oil industry. In some embodiments, the presence of a significant amount of polyunsaturated fatty acid in the oil will provide greater flexibility and variety for the production of hydrocarbons, since the multiple sites of unsaturation in a polyunsaturated fatty acid provide multiple sites for cleavage to make hydrocarbons. For example, certain jet fuels require hydrocarbons with two to eight carbons. Polyunsaturated fatty acids can be cleaved through known processes in the art, such as cracking, to produce shorter hydrocarbons of various chain lengths.

In some embodiments, the biofuel is produced by thermal depolymerization. In some embodiments, the biofuel is a renewable diesel. As used herein, thermal depolymerization includes any process for the production of renewable diesel using superheated water.

In some embodiments, the biofuel is produced by adding the oil to a petroleum refining process during the production of diesel fuel. In some embodiments, the biofuel is a co-processed renewable diesel.

Having generally described this invention, a further understanding can be obtained by reference to the examples provided herein. These examples are for purposes of illustration only and are not intended to be limiting.

### Example 1

Construction of the pCL0076 Expression Vector

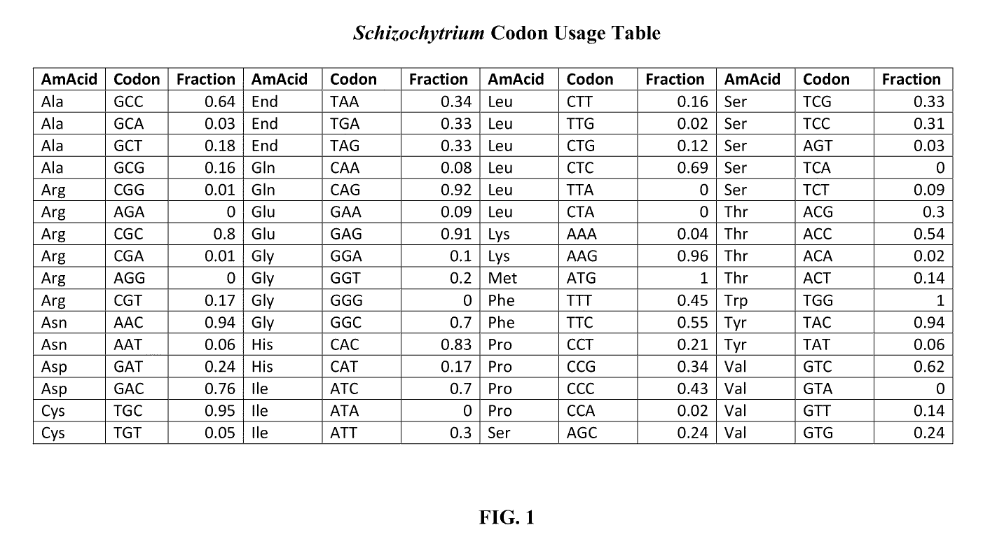

The vector pAB0018 (ATCC Accession No. PTA-9616) was digested with BamHI and NdeI resulting in two fragments of 838 bp and 9879 bp in length. The 9879 bp fragment was fractionated by standard electrophoretic techniques in an agar gel, purified using commercial DNA purification kits, and ligated to a sequence (SEQ ID NO:1) comprising a polynucleotide sequence encoding the native secretion signal of the Sec1 protein of Schizochytrium sp. ATCC number 20888 followed by a synthetic sequence encoding the mature invertase protein (SUC2) of Saccharomyces cerevisiae, codon-optimized using the Schizochytrium codon usage table in FIG. 1 (codon optimization was performed by Blue Heron Biotechnology, Bothell, Wash.). A fusion sequence containing the sequences encoding the Schizochytrium Sec1 signal peptide and the Saccharomyces cerevisiae SUC2 secreted invertase protein (GenBank Accession No. P00724) was inserted into the Schizochytrium vector pSchiz, followed by digestion with BamHI and NdeI to yield SEQ ID NO:1.

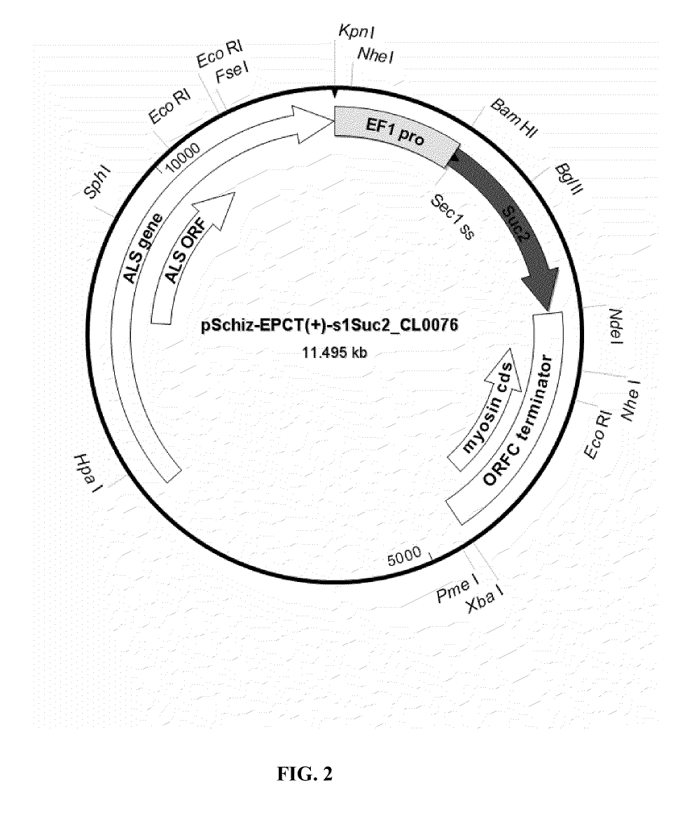

The ligation product was then used to transform a commercially supplied strain of competent DH5α E. coli cells (Invitrogen) using the manufacturer's protocols. Several of the resulting clones were propagated and their plasmids were extracted and purified. These plasmids were then screened by restriction digests and/or PCR to confirm that the ligation generated the expected plasmid vectors. One such plasmid vector resulting from the ligation of the 9879 bp fragment and SEQ ID NO:1 was verified by Sanger sequencing and designated pCL0076 (SEQ ID NO:2). See FIG. 2.

### Example 2

### Growth of Schizochytrium on Sucrose

Cultures of Schizochytrium sp. ATCC 20888 and a genetically modified Schizochytrium derivative, designated B76-32, were grown in M2B medium consisting of 10 g/L glucose, 0.8 g/L (NH₄)₂SO₄, 5 g/L Na₂SO₄, 2 g/L MgSO₄.7H₂O, 0.5 g/L KH₂PO₄, 0.5 g/L KCl, 0.1 g/L CaCl₂.2H₂O, 0.1 M MES (pH 6.0) 0.1% PB26 metals, and 0.1% PB26 Vitamins (v/v). PB26 vitamins consisted of 50 mg/mL vitamin B12, 100 μg/mL thiamine, and 100 μg/mL Ca-pantothenate. PB26 metals were adjusted to pH 4.5 and consisted of 3 g/L FeSO₄.7H₂O, 1 g/L MnCl₂.₄H₂O, 800 mg/mL ZnSO₄.7H₂O, 20 mg/mL CoCl₂.6H₂O, 10 mg/mL Na₂MoO₄.2H₂O, 600 mg/mL CuSO₄.5H₂O, and 800 mg/mL NiSO₄.6H₂O. PB26 stock solutions were filter sterilized separately and added to the broth after autoclaving. Glucose, KH₂PO₄, and CaCl₂.2H₂O were each autoclaved separately from the remainder of the broth ingredients before mixing to prevent salt precipitation and carbohydrate caramelizing. All medium ingredients were purchased from Sigma Chemical (St. Louis, Mo.). Strain B76-32 is a derivative of Schizochytrium sp. ATCC 20888 engineered according to U.S. Pat. No. 7,211,418 and U.S. Publ. Nos. 2008/0022422 and 2008/0026434.

Cultures of Schizochytrium sp. ATCC 20888 and B76-32 were grown to log phase and were transformed with the vector pCL0076 using electroporation with enzyme pretreatment as described below.

Electroporation with enzyme pretreatment—Cells were grown in 50 mL of M50-20 media (see U.S. Publ. No. 2008/0022422) on a shaker at 200 rpm for 2 days at 30° C. The cells were diluted at 1:100 into M2B media (see following paragraph) and grown overnight (16-24 h), attempting to reach mid-log phase growth (OD600 of 1.5-2.5). The cells were centrifuged in a 50 mL conical tube for 5 min at about 3000×g. The supernatant was removed and the cells were resuspended in 1 M mannitol, pH 5.5, in a suitable volume to reach a final concentration of 20D₆₀₀ units. 5 mL of cells were aliquoted into a 25 mL shaker flask and amended with 10 mM CaCl₂ (1.0 M stock, filter sterilized) and 0.25 mg/mL Protease XIV (10 mg/mL stock, filter sterilized; Sigma-Aldrich, St. Louis, Mo.). Flasks were incubated on a shaker at 30° C. and about 100 rpm for 4 h. Cells were monitored under the microscope to determine the degree of protoplasting, with single cells desired. The cells were centrifuged for 5 min at about 2500×g in round-bottom tubes (i.e., 14 mL Falcon™ tubes, BD Biosciences, San Jose, Calif.). The supernatant was removed and the cells were gently resuspended with 5 mL of ice cold 10% glycerol. The cells were re-centrifuged for 5 min at about 2500×g in round-bottom tubes. The supernatant was removed and the cells were gently resuspended with 500 μL of ice cold 10% glycerol, using wide-bore pipette tips. 90 μL of cells were aliquoted into a prechilled electro-cuvette (Gene Pulser® cuvette—0.1 cm gap or 0.2 cm gap, Bio-Rad, Hercules, Calif.). One μg to 5 μg of DNA (in less than or equal to a 10 μA volume) was added to the cuvette, mixed gently with a pipette tip, and placed on ice for 5 min. Cells were electroporated at 200 ohms (resistance), 25 μF (capacitance), and either 250V (for 0.1 cm gap) or 500V (0.2 cm gap). 0.5 mL of M50-20 media was added immediately to the cuvette. The cells were then transferred to 4.5 mL of M50-20 media in a 25 mL shaker flask and incubated for 2-3 h at 30° C. and about 100 rpm on a shaker. The cells were centrifuged for 5 min at about 2500×g in round bottom tubes. The supernatant was removed and the cell pellet was resuspended in 0.5 mL of M50-20 media. Cells were plated onto an appropriate number (2 to 5) of M2B plates with appropriate selection (if needed) and incubated at 30° C.

Transformants were selected for growth in either M2B+SMM media or directly selected for growth on sucrose by plating onto MSFM+sucrose. For MSFM+sucrose selection, after 1-2 weeks colonies were replated with several passes onto fresh sucrose-containing media. It was determined that expression of invertase can be used as a selectable marker for thraustochytrid colonies grown on sucrose as a sole carbon source.

For the following experiments, primary transformants were selected for growth on solid M2B media containing 20 g/L agar (VWR, West Chester, Pa.) and 10 μg/mL SMM (Chem Service, Westchester, Pa.) after 2-6 days of incubation at 27° C. All primary transformants were manually transferred to fresh M2B plates with SMM. After 1 week the colonies were transferred to MSFM and 5 g/L sucrose without SMM. After 1 week the largest colonies were transferred to fresh MSFM/sucrose media plates. Ten of the Schizochytrium sp. ATCC 20888 transformants growing on sucrose were selected for further characterization and were designated as 1-1, 1-3, 1-24, 3-1, 3-2, 3-5, 3-21, 4-1, 4-24, and 4-31, respectively. Nine of the B76-32 transformants growing on sucrose were selected for further characterization and were designated as B76-32 #2, #12, #19, #26, #30, #39, #42, #56, and #61.

Colonies growing on sucrose (1-1, 1-3, 1-24, 3-1, 3-2, 3-5, 3-21, 4-1, 4-24, 4-31) were removed from plates using an inoculation loop and transferred into culture tubes containing 5 mL of sucrose media and grown for 4 days at 29° C. on a shaker. 2 mL of this culture was used to inoculate 50 mL of media (MSFM or SSFM) in 250 ml flasks and grown at 29° C. on a shaker at 200 rpm.

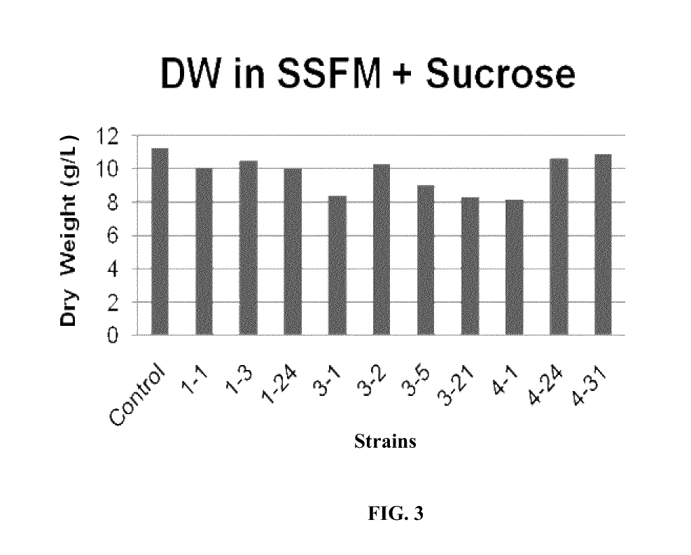

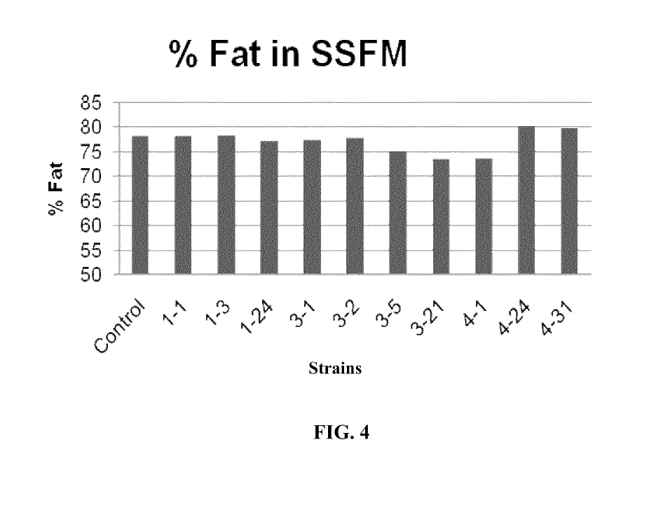

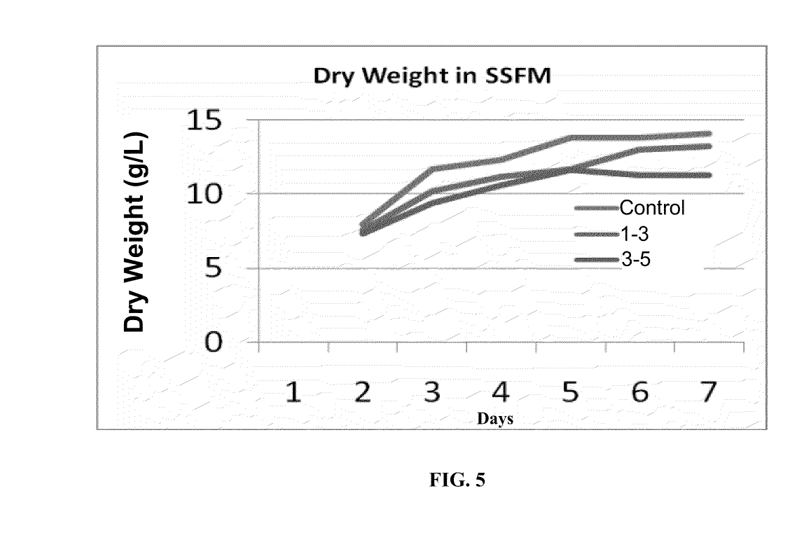

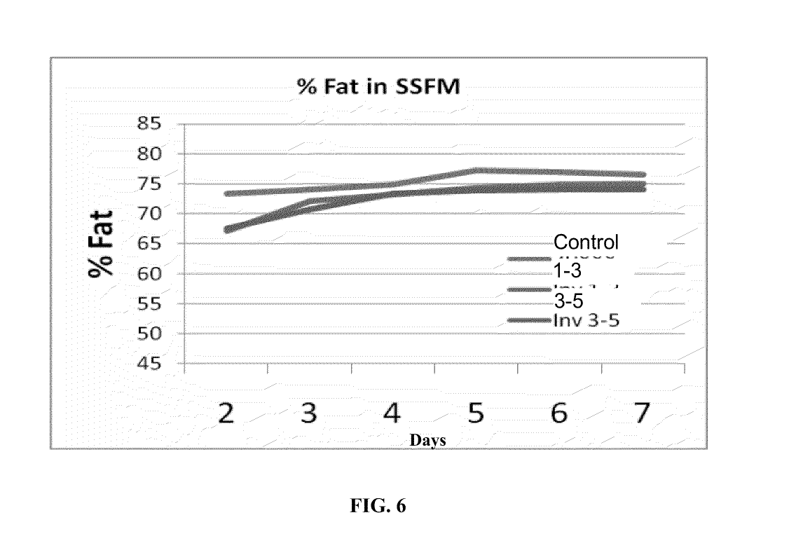

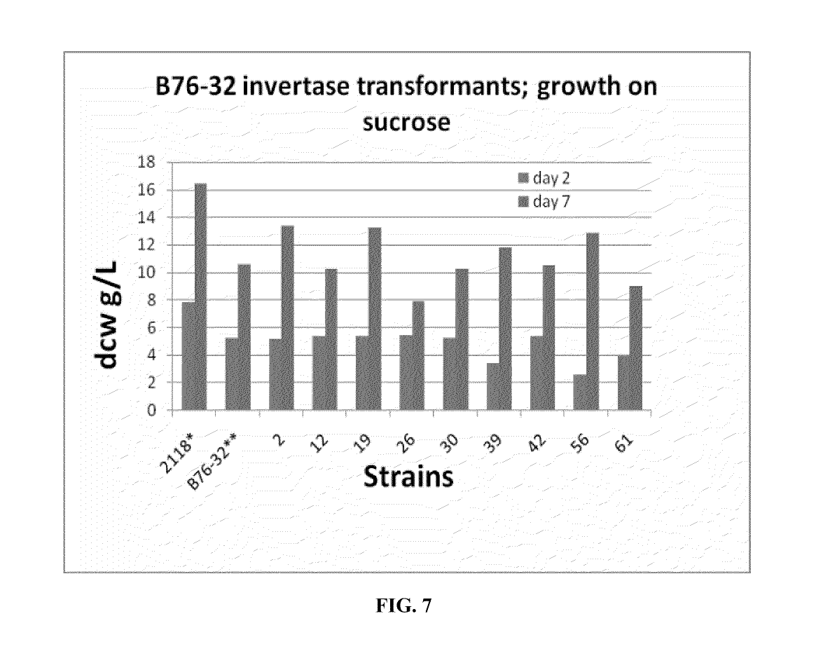

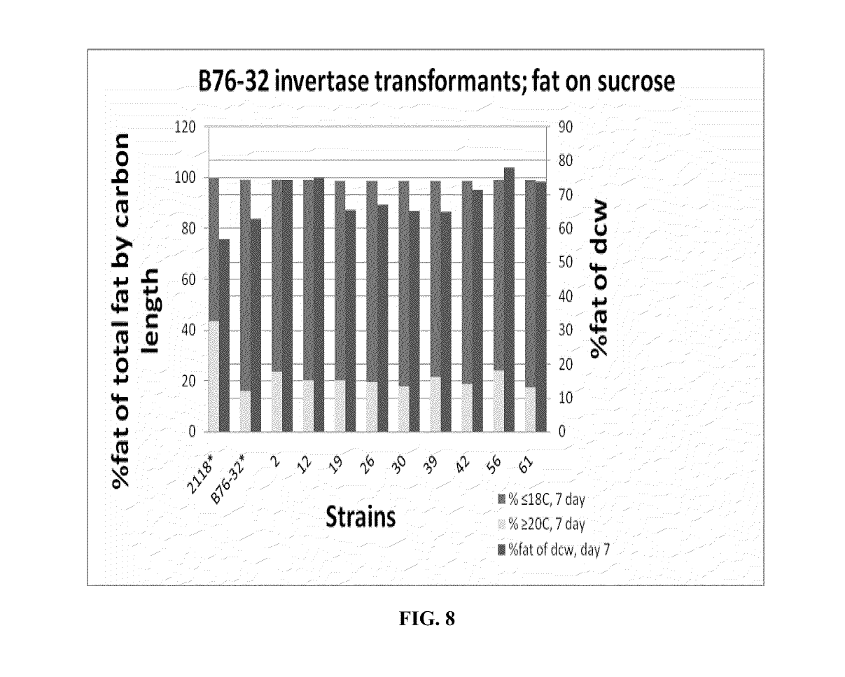

Control flasks of the parental strain Schizochytrium sp. ATCC 20888 were grown the same way but using glucose containing media. Cells were harvested after 7 days. Cells were centrifuged and washed with a 50% isopropanol:distilled water mixture. The pelleted cells were freeze-dried, weighed, and a fatty acid methyl esters (FAME) analysis was performed. For FAME analysis, oils were extracted from cell cultures using standard procedures and analyzed for fatty acid composition as a percent of total fatty acid methyl esters (FAMEs). See, e.g., U.S. Publ. No. 2010/0239533, incorporated by reference in its entirety. Growth and fat content of CL0076 transformants of Schizochytrium sp. ATCC 20888 or B76-32 were assayed gravimetrically and by gas chromatography of derivatized oils as previously described in U.S. Publ. No. 2008/0022422, incorporated herein by reference in its entirety. Results are shown in Tables 5-8. Dry weights and fat content of pellets from shake flask cultures of transformants as well as parent strains are shown in FIGS. 3-8.

SSFM media: 50 g/L glucose or sucrose, 13.6 g/L Na₂SO₄, 0.7 g/L K₂SO₄, 0.36 g/L KCl, 2.3 g/L MgSO₄.7H₂O, 0.1M MES (pH 6.0), 1.2 g/L (NH₄)₂SO₄, 0.13 g/L monosodium glutamate, 0.056 g/L KH₂PO₄, and 0.2 g/L CaCl₂.2H₂O. Vitamins were added at 1 mL/L from a stock consisting of 0.16 g/L vitamin B12, 9.7 g/L thiamine, and 3.3 g/L Ca-pantothenate. Trace metals were added at 2 mL/L from a stock consisting of 1 g/L citric acid, 5.2 g/L FeSO₄.7H₂O, 1.5 g/L MnCl₂.₄H₂O, 1.5 g/L ZnSO₄.7H₂O, 0.02 g/L CoCl₂.6H₂O, 0.02 g/L Na₂MoO₄.2H₂O, 1.0 g/L CuSO₄.5H₂O, and 1.0 g/L NiSO₄.6H₂O, adjusted to pH 2.5.

Modified SFM (MSFM) media: 10 g/L glucose or sucrose, 25.0 g/L NaCl, 1.0 g/L KCl, 0.2 g/L (NH4)2SO4, 5 g/L, 5.0 g/L MgSO4.7H2O, 0.1 g/L KH2PO4, 0.3 g/L CaCl2.2H2O, 0.1 M HEPES (pH 7.0), 0.1% PB26 metals, and 0.1% PB26 Vitamins (v/v). Vitamins were added at 2 mL/L from a stock consisting of 0.16 g/L vitamin B12, 9.7 g/L thiamine, and 3.3 g/L Ca-pantothenate. Trace metals were added at 2 mL/L from a stock consisting of 1 g/L citric acid, 5.2 g/L FeSO4.7H2O, 1.5 g/L MnCl2.4H2O, 1.5 g/L ZnSO4.7H2O, 0.02 g/L CoCl2.6H2O, 0.02 g/L Na2MoO4.2H2O, 1.0 g/L CuSO4.5H2O, and 1.0 g/L NiSO4.6H2O, adjusted to pH 2.5.

Table 5 shows the growth and fat levels of Schizochytrium sp. ATCC 20888 grown in MSFM with glucose, fructose, sucrose, or no added carbon source.

Table 6 shows the dry weight and % fatty acid for Schizochytrium sp. ATCC 20888 grown in MSFM media with glucose (control) and Schizochytrium sp. ATCC 20888 transformed cell lines grown in MSFM media with sucrose.

Table 7 shows the dry weight and % fatty acid for Schizochytrium sp. ATCC 20888 grown in SSFM media with glucose (control) and Schizochytrium sp. ATCC 20888 transformed cell lines grown in SSFM media with sucrose.

Table 8 shows the dry weight and % fatty acid for Schizochytrium B76-32 grown in SSFM media with glucose (control) and Schizochytrium B76-32 transformed cell lines grown in SSFM media with sucrose.

TABLE 5
Growth and fat levels of Schizochytrium sp. ATCC 20888 grown in
MSFM with glucose, fructose, sucrose or no added carbon source.

| | Glucose | Fructose | Sucrose | No added carbon |
|---|---|---|---|---|
| DW (g/L) | 2.84 | 2.65 | 0.16 | 0.11 |
| % FA | 66.5 | 65.3 | ND | ND |

DW = Dry Weight
FA = Fatty Acids

TABLE 6
Schizochytrium sp. ATCC 20888 transformed cell lines
grown in MSFM media with sucrose.

| | 20888 | | | | | | | | |
|---|---|---|---|---|---|---|---|---|---|
| | control | 1-1 | 1-3 | 3-2 | 3-5 | 3-21 | 4-1 | 4-24 | 4-31 |
| DW | 2.94 | 2.49 | 2.79 | 2.21 | 2.60 | 2.64 | 2.44 | 3.05 | 2.24 |
| (g/L) | | | | | | | | | |
| % FA | 70.87 | 70.79 | 72.36 | 67.97 | 69.78 | 71.05 | 68.84 | 73.85 | 73.66 |

DW = Dry Weight
FA = Fatty Acids

TABLE 7
Schizochytrium sp. ATCC 20888 transformed cell lines grown in SSFM
media with sucrose.

| | 20888 | | | | | | | | | | |
|---|---|---|---|---|---|---|---|---|---|---|---|
| | control | 1-1 | 1-3 | 1-24 | 3-1 | 3-2 | 3-5 | 3-21 | 4-1 | 4-24 | 4-31 |
| DW | 11.24 | 10.04 | 10.51 | 9.99 | 8.40 | 10.29 | 9.03 | 8.34 | 8.16 | 10.63 | 10.92 |
| (g/L) | | | | | | | | | | | |
| % FA | 78.22 | 78.20 | 76.29 | 77.10 | 77.37 | 77.71 | 74.97 | 73.44 | 73.65 | 80.05 | 79.82 |

DW = Dry Weight
FA = Fatty Acids

TABLE 8
B76-32 transformed cell lines grown in SSFM media with sucrose.

| | B76-32 | | | | | | | | | |
|---|---|---|---|---|---|---|---|---|---|---|
| | control | #2 | #12 | #19 | #26 | #30 | #39 | #42 | #56 | #61 |
| 7-day | 10.56 | 13.37 | 10.21 | 13.26 | 7.88 | 10.26 | 11.81 | 10.47 | 12.84 | 8.97 |
| DW (g/L) | | | | | | | | | | |
| % FA | 62.8 | 74.3 | 75.2 | 65.4 | 66.9 | 65.1 | 64.8 | 71.4 | 77.9 | 73.7 |

DW = Dry Weight
FA = Fatty Acids

### Example 3

### Expression of Invertase in Schizochytrium

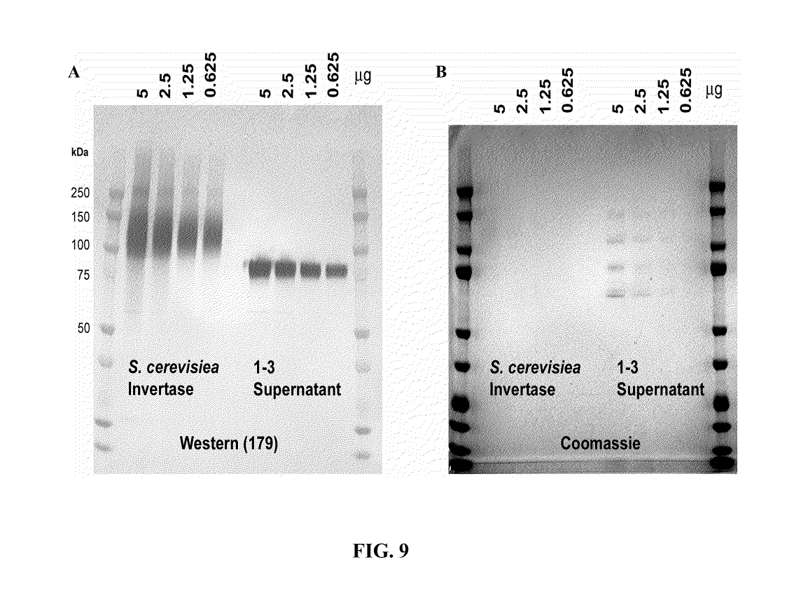

Cell-free supernatants of 50 mL shake-flask cultures of Schizochytrium sp. ATCC 20888 clone 1-3 transformed with pCL0076 (Example 2) grown in SSFM for 3 days (see U.S. Publ. No. 2008/0022422) were collected after cultures were centrifuged at 5000×g. Culture supernatants were used either directly for SDS-PAGE or were concentrated 50 to 100-fold using commercially available concentrators equipped with permeable membranes permitting concentration of all components heavier than 10 kDa. Total protein concentration was measured by Bradford assay (Biorad). The expression of invertase was then verified by immunoblot analysis following standard immunoblotting procedure (Sambrook et al.). Briefly, the proteins (0.625 μg to 5 μg) were separated by SDS-PAGE on a bis-tris gel (Invitrogen, Carlsbad, Calif., USA). The proteins were then stained with Coomassie blue (SimplyBlue Safe Stain, Invitrogen, Carlsbad, Calif., USA) or transferred onto polyvinylidene fluoride membrane and probed for the presence of invertase protein with an invertase antisera (Open Biosystems, Huntsville, Ala.) derived from rabbits that had been injected with a pure preparation of Saccharomyces cerevisiae invertase (Sigma, St. Louis, Mo.). The membrane was subsequently incubated with a mouse anti-rabbit secondary antibody coupled to alkaline phosphatase (Promega Corporation, Madison, Wis.). The membrane was then treated with 5-bromo-4-chloro-3-indoyl-phosphate/nitroblue tetrazolium solution (BCIP/NBT) according to the manufacturer's instructions (KPL, Gaithersburg, Md.). An example is presented in FIG. 9. Anti-invertase immunoblot and corresponding Coomassie blue-stained gel are presented in panels 9A and 9B, respectively. Of the four major bands seen in culture supernatants of clone 1-3, only one was shown to react with anti-invertase antisera. The identity of the protein was confirmed by peptide sequence analysis.

### Example 4

### Invertase Activity in Schizochytrium

Sucrase activity was measured by the rate of liberation of fructose and glucose from sucrose. The assay was performed by adding sucrose to fermentation broth supernatant and the glucose/fructose content was measured by HPLC.

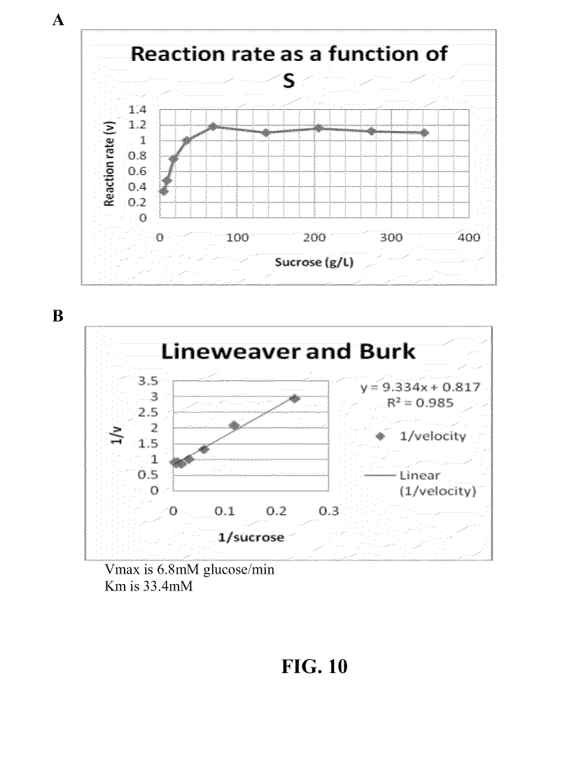

Schizochytrium sp. B76-32 clone #2 transformed with pCL0076 (Example 2) was grown in MSFM (with sucrose) until the OD₆₀₀ₙₘ reached about 4 in 50 mL shake flasks at 29° C. Cells were spun down for 15 min at 4500×g and invertase activity was measured in the supernatant. Invertase was assayed by adding 0.1 M sucrose to varying volumes of fermentation broth and adjusting the final volume to 1 mL. The reaction was incubated at 55° C. for 3 min. Termination of the reaction was done at 100° C. for 10 min, and then frozen prior to quantification of glucose, fructose, and sucrose by HPLC. HPLC was performed using a modified version of the process described in Liu et al., Food Sci. 28:293-296 (2007). Briefly, mono- and di-saccharides were separated using an HPLC with a Luna NH₂ column and detected using an RID (refractive index detector). Identification was carried out by comparing retention times to those of standards. Quantitation was by an external standard calibration. The reaction rate as a function of sucrose concentration is shown in FIG. 10A. The Km (33.4 mM) and Vmax (6.8 mM glucose/min) were calculated from a standard Lineweaver-Burk plot. See FIG. 10B.

### Example 5

Glycosylation of Invertase in Schizochytrium

Supernatant proteins from Example 4 were separated by SDS-PAGE on a 4-12% bis-tris gel (Invitrogen). The proteins were then stained with Coomassie blue (SimplyBlue™ Safe Stain, Invitrogen, Carlsbad, Calif.). The stained proteins of interest were cut from the gel and slices cut into smaller pieces (˜1 mm³) and destained alternately with 40 mM Ammonium bicarbonate (AmBic) and 100% acetonitrile until the color turned clear. Destained gel was reswelled in 10 mM DTT in 40 mM Ambic at 55° C. for 1 h. The DTT solution was exchanged with 55 mM Iodoacetamide (IAM) and incubated in the dark for 45 min. Incubation was followed by washing alternately with 40 mM AmBic and 100% acetonitrile twice. Dehydrated gel was reswelled with trypsin solution (trypsin in 40 mM Ambic) on ice for 45 min initially, and protein digestion was carried out at 37° C. overnight. The supernatant was transferred into another tube. Peptides and glycopeptides were extracted from the gel in series with 20% acetonitrile in 5% formic acid, 50% acetonitrile in 5% formic acid, and then 80% acetonitrile in 5% formic acid. The sample solutions were dried and combined into one tube. Extracted tryptic digest was passed through a C18 Sep-Pak® cartridge (Waters Corporation, Milford, Mass.) and washed with 5% acetic acid to remove contaminants (such as salts and SDS). Peptides and glycopeptides were eluted in series with 20% isopropanol in 5% acetic acid, 40% isopropanol in 5% acetic acid, and 100% isopropanol and were dried in a speed vacuum concentrator. The dried samples were combined and then reconstituted with 50 mM sodium phosphate buffer (pH 7.5) and heated at 100° C. for 5 min to inactivate trypsin. The tryptic digest was incubated with PNGase F at 37° C. overnight to release N-glycans. After digestion, the sample was passed through a C18 Sep-Pak® cartridge and the carbohydrate fraction was eluted with 5% acetic acid and dried by lyophilization. Released N-linked oligosaccharides were permethylated based on the method of Anumula and Taylor (Anumula and Taylor, 1992) and profiled by mass spectrometry. Mass spectrometric analysis was performed following the method developed at the Complex Carbohydrates Research Center (Aoki K et al., J. Biol. Chem. 282:9127-42 (2007). Mass analysis was determined by using NSI-LTQ/MSₙ. Briefly, permethylated glycans were dissolved in 1 mM NaOH in 50% methanol and infused directly into the instrument (Finnigan™ LTQ™ Linear Ion Trap Mass Spectrometer, Thermo Electron Corporation, Waltham, Mass.) at a constant flow rate of 0.4 μL/min. The MS analysis was performed in the positive ion mode. For total ion mapping, automated MS/MS analysis (at 35 collision energy), m/z range from 500 to 2000 was scanned in successive 2.8 mass unit windows that overlapped the preceding window by 2 mass units.

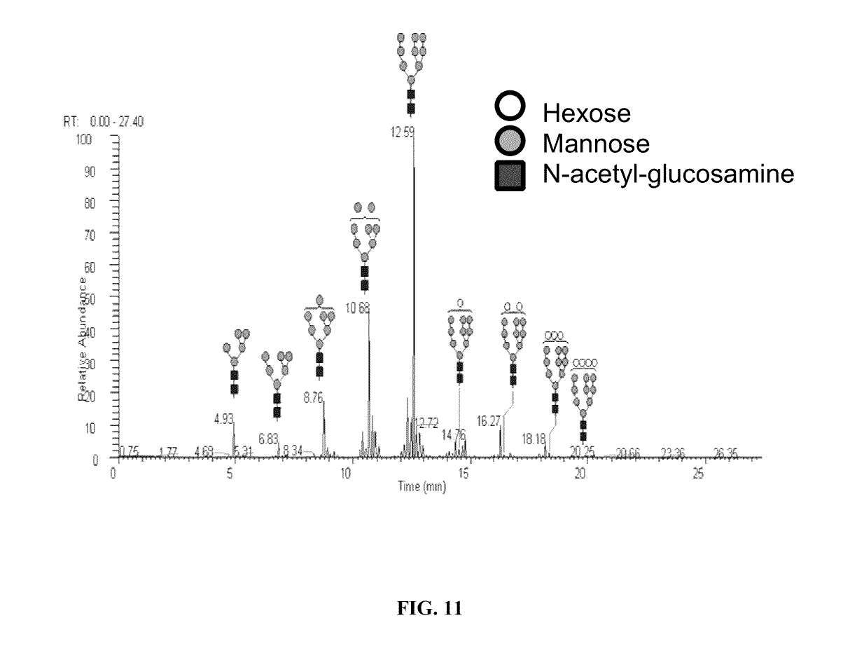

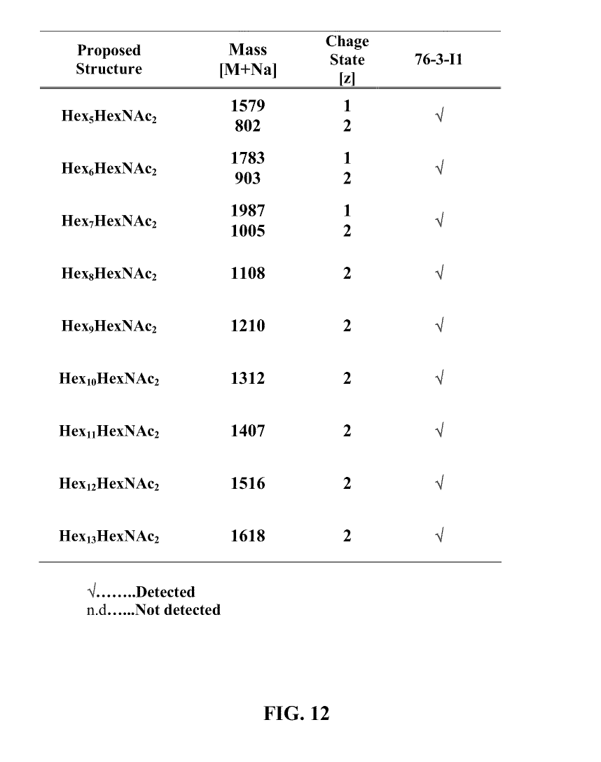

Total ion mapping was performed to examine the presence of fragment ions indicative of glycans. All MS/MS data from m/z 500 through m/z 2000 were taken and the raw data were analyzed manually. The chromatogram and table of species obtained by NSI-total ion mapping are shown in FIG. 11 and FIG. 12. This chromatogram was processed by the scan filter, neutral loss of m/z 139, which is the one of characteristic neutral loss of high-mannose type glycans. Total ion mapping revealed that this sample contains a series of high-mannose type glycans with long mannose chains.

### Example 6

Expression of Aspergillus niger Invertase in Schizochytrium

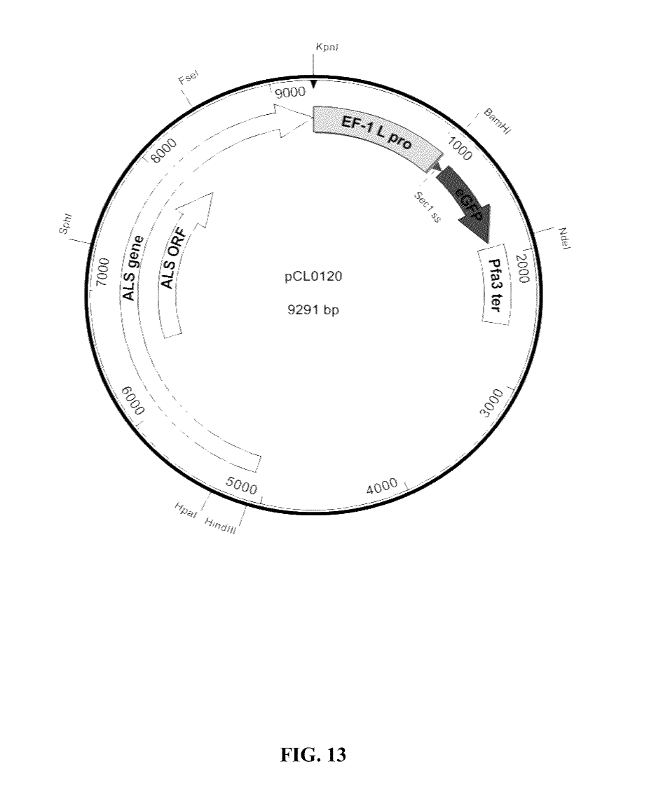

The vector pAB0018 (ATCC Accession No. PTA-9616) was digested with HindIII, treated with mung bean nuclease, purified, and then further digested with KpnI generating four fragments of various sizes. A fragment of 2552 bp was isolated by standard electrophoretic techniques in an agar gel and purified using commercial DNA purification kits. A second digest of pAB0018 with PmeI and Kpn was then performed. A fragment of 6732 bp was isolated and purified from this digest and ligated to the 2552 bp fragment. The ligation product was then used to transform commercially supplied strains of competent DH5-α E. coli cells (Invitrogen) using the manufacturer's protocol. Plasmids from ampicillin-resistant clones were propagated, purified, and then screened by restriction digests or PCR to confirm that the ligation generated the expected plasmid structures. One verified plasmid was designated pCL0120. See FIG. 13 and SEQ ID NO:5.

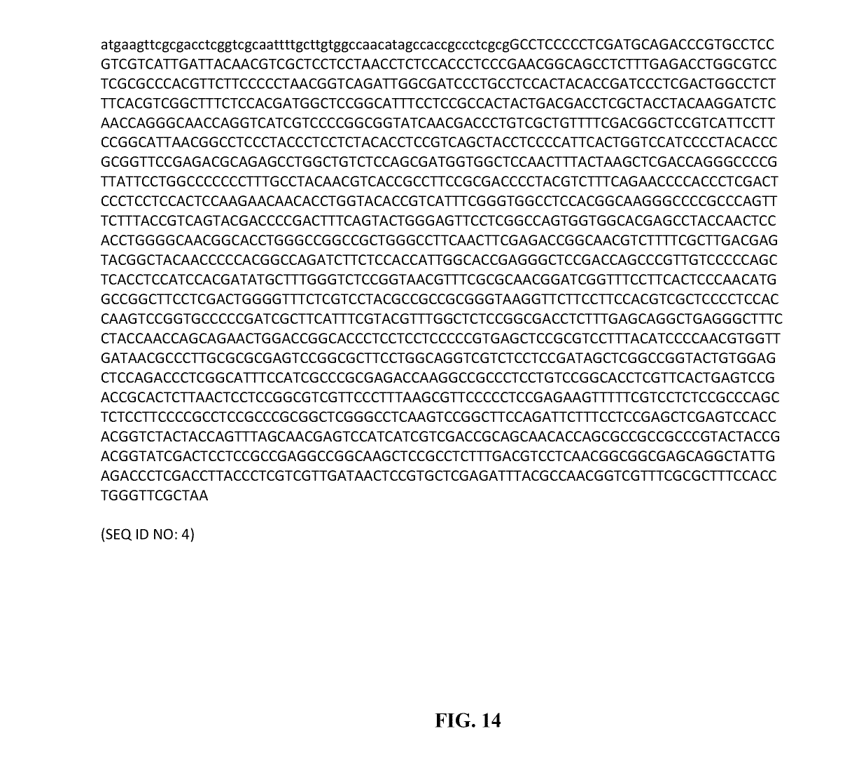

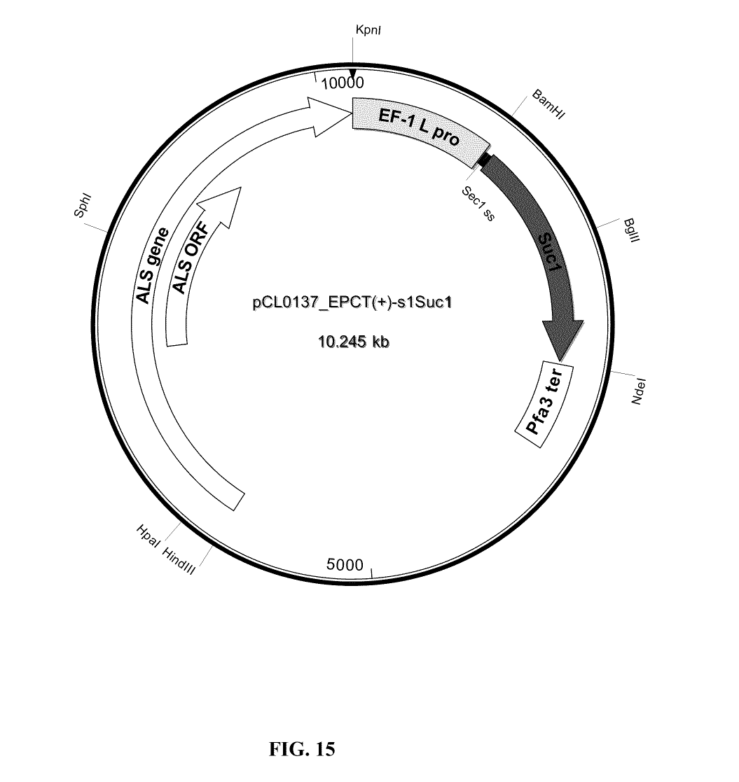

The polynucleotide sequence encoding the mature form of the Suc1 secreted invertase protein from the fungus Aspergillus niger (GenBank Accession No. 533920) was codon-optimized for expression in Schizochytrium using the Schizochytrium codon usage table of FIG. 1 (codon optimization was performed by Blue Heron Biotechnology, Bothell, Wash.). The codon-optimized sequence was synthesized and the resulting polynucleotide sequence was fused to a polynucleotide sequence encoding the Schizochytrium Sec1 signal peptide (“Sec1 ss”) as an N-terminal leader in place of the endogenous signal peptide. The resulting coding region of the “s1Suc1” nucleic acid sequence (SEQ ID NO:4) is shown in FIG. 14. This codon-optimized s1Suc1 polynucleotide was cloned to the vector pCL0120 using the 5′ and 3′ restriction sites BamHI and NdeI for insertion and ligation according to standard techniques. A plasmid map of the resulting vector, pCL0137, is shown in FIG. 15. Wild-type strain Schizochytrium sp. ATCC 20888 was transformed with this vector and the resulting clones were selected on solid SSFM media containing SMM. SMM-resistant clones were then re-plated to SSFM solid media containing sucrose as a sole carbon source to assay for growth. Depending on the transformation experiment, between 50% and 90% of the SMM-resistant primary transformants were capable of growth on sucrose media.

### Example 7

### Expression of Secreted Invertase, Cytoplasmic Invertase, and Sucrose Transporter in Schizochytrium

The Saccharomyces cerevisiae AGT1 alpha-glucoside transporter gene (GenBank Accession No. L47346) was codon-optimized for expression in Schizochytrium and was inserted into the vector pCL0121 (SEQ ID NO:6), comprising a ZEOCIN™ selectable marker, to produce the vector pCL0125 (SEQ ID NO:7).

The Solanum tuberosum (potato) SUT1 sucrose transporter gene (GenBank Accession No. X69165) was codon-optimized for expression in Schizochytrium and was inserted into the vector pCL0121 (SEQ ID NO:6), comprising a ZEOCIN™ selectable marker, to produce the vector pCL0126 (SEQ ID NO:8).

The polynucleotide sequence encoding the Saccharomyces cerevisiae cytoplasmic

SUC2 invertase protein (GenBank Accession No. P00724) was codon-optimized for expression in Schizochytrium and was inserted into the vector pCL0122 (SEQ ID NO:9), comprising a paromomycin selectable marker, to produce the vector pCL0127 (SEQ ID NO:10).

Schizochytrium sp. B76-32 clone #19 transformed with pCL0076 was further transformed with either pCL0125 or pCL0126, comprising the AGT1 alpha-glucoside transporter and SUT1 sucrose transporter, respectively. Transformants were selected on antibiotics and the presence of the transporter genes was confirmed by PCR. Transformants were further transformed with pCL0127 comprising cytoplasmic SUC2, were selected on antibiotic, and were analyzed for growth on sucrose based on dry weight and FAME content as described in Example 2. Growth on sucrose and production of fatty acids were observed.

Wild type Schizochytrium sp. ATCC 20888 cells were transformed with either: (1) pCL0125, comprising the AGT1 alpha-glucoside transporter, in combination with pCL0127, comprising cytoplasmic SUC2; or (2) pCL0126, comprising the SUT1 sucrose transporter, in combination with pCL0127, comprising cytoplasmic SUC2. Transformants were grown and selected on sucrose. Each of the combinations of pCL0125/pCL0127 and pCL0126/pCL0127 produced transformants capable of growth on sucrose. Positive transformants were identified and frozen. At a subsequent date, the positive transformants are thawed, are further transformed with pCL0076 comprising secreted invertase, are selected on antibiotic, and are analyzed for growth levels and FAME as described in Example 2.

Schizochytrium sp. ATCC 20888 transformed with pCL0076 comprising the SUC2 secreted invertase (clone 1-3, Example 2) was further transformed with either: (1) pCL0125, comprising the AGT1 alpha-glucoside transporter, in combination with pCL0127, comprising cytoplasmic SUC2; or (2) pCL0126, comprising the SUT1 sucrose transporter, in combination with pCL0127, comprising cytoplasmic SUC2. Transformants were grown and selected on ZEOCIN™ and paromomycin. Positive transformants were identified and frozen. At a subsequent date, the positive transformants are thawed and are analyzed for growth on sucrose and for fatty acid methyl esters as described in Example 2.

All of the various aspects, embodiments, and options described herein can be combined in any and all variations.

All publications, patents, and patent applications mentioned in this specification are herein incorporated by reference to the same extent as if each individual publication, patent, or patent application was specifically and individually indicated to be incorporated by reference.

1. A method of producing a thraustochytrid cell culture, comprising:
(a) transforming a thraustochytrid cell with a nucleic acid molecule comprising a polynucleotide sequence encoding a heterologous sucrase,
(b) growing the transformed thraustochytrid cell in a culture medium comprising sucrose as a carbon source.

2. The method of claim 1, wherein the sucrase is an invertase.

3. The method of claim 1, wherein the sucrase is operably linked to a signal peptide.

4. The method of claim 1, wherein the polynucleotide sequence encoding the heterologous sucrase is at least 90% identical to a sequence selected from the group consisting of: a polynucleotide sequence encoding the amino acid sequence of Accession No. P00724 or S33920; a polynucleotide sequence encoding the amino acid sequence of Accession No. P00724 or S33920, wherein the polynucleotide sequence is codon-optimized for expression in Schizochytrium; and the polynucleotide sequence of SEQ ID NO:1 or SEQ ID NO:4.

5. The method of claim 1, further comprising transforming the thraustochytrid cell with a nucleic acid molecule comprising a polynucleotide sequence encoding a heterologous sucrose transporter.

6. The method of claim 5, wherein the polynucleotide sequence encoding the heterologous sucrose transporter is at least 90% identical to a sequence selected from the group consisting of: a polynucleotide sequence of Accession No. L47346 or X69165; and a polynucleotide sequence of Accession No. L47346 or X69165 that is codon-optimized for expression in Schizochytrium.

7. The method of claim 1, wherein the thraustochytrid is a Schizochytrium or a Thraustochytrium.

8. A thraustochytrid cell culture produced by the method of claim 1.

9. A thraustochytrid cell comprising a nucleic acid molecule comprising a polynucleotide sequence encoding a heterologous sucrase.

10. The cell of claim 9, wherein the sucrose is an invertase.

11. The cell according to claim 9, wherein the sucrase is operably linked to a signal peptide.

12. The cell of claim 9, wherein the polynucleotide sequence encoding the heterologous sucrase is at least 90% identical to a sequence selected from the group consisting of: a polynucleotide sequence encoding the amino acid sequence of Accession No. P00724 or S33920; a polynucleotide sequence encoding the amino acid sequence of Accession No. P00724 or S33920, wherein the polynucleotide sequence is codon-optimized for expression in Schizochytrium; and the polynucleotide sequence of SEQ ID NO: 1 or SEQ ID NO: 4.

13. The cell of claim 9, wherein the cell further comprises a nucleic acid molecule compromising a polynucleotide sequence encoding a heterologous sucrose transporter.

14. The cell of claim 13, wherein the polynucleotide sequence encoding the heterologous sucrose transporter is at least 90% identical to a sequence selected from the group consisting of: a polynucleotide sequence of Accession No. L47346 or X69165; and a polynucleotide sequence of Accession No. L47346 or X69165 that is codon-optimized for expression in Schizochytrium.

15. The cell of claim 9, wherein the thraustochytrid is a Schizochytrium or a Thraustochytrium.

16. A thraustochytrid culture comprising:
(a) the thraustochytrid cell of claim 9, and
(b) a cell culture medium comprising sucrose as a carbon source.

17. A method of producing a thraustochytrid biomass, comprising:
(a) growing the thraustochytrid cell of claim 9 in a culture medium comprising sucrose as a carbon source, and
(b) harvesting the biomass from the culture medium.

18. A method of producing a microbial oil, comprising:
(a) growing the thraustochytrid cell of claim 9 in a culture medium comprising sucrose as a carbon source to produce a biomass, and
(b) extracting an oil from the biomass.

19. A method of producing a food product, cosmetic, industrial composition, or pharmaceutical composition for an animal or human, comprising:
(a) growing the thraustochytrid cell of claim 9 culture medium comprising sucrose as a carbon source,
(b) harvesting a biomass from the culture medium, and
(c) preparing the food product, cosmetic, industrial composition, or pharmaceutical composition from the biomass.

20. The method of claim 19, further comprising extracting an oil from the biomass and preparing the food product, cosmetic, industrial composition, or pharmaceutical composition from the oil.

21. The method of claim 19, wherein the food product is an infant formula.

22. The method of claim 21, wherein the infant formula is suitable for premature infants.

23. The method of claim 19, wherein the food product is milk, a beverage, a therapeutic drink, a nutritional drink, or a combination thereof.

24. The method of claim 19, wherein the food product is an additive for animal or human food.

25. The method of claim 19, wherein the food product is a nutritional supplement.

26. The method of claim 19, wherein the food product is an animal feed.

27. The method of claim 26, wherein the animal feed is an aquaculture feed.

28. The method of claim 26, wherein the animal feed is a domestic animal feed, a zoological animal feed, a work animal feed, a livestock feed, or a combination thereof.

29. A method for producing a biofuel, comprising:
(a) growing the thraustochytrid cell of claim 9 culture medium comprising sucrose as a carbon source to produce a biomass,
(b) extracting an oil from the biomass, and
(c) producing a biofuel by transesterifying the oil, cracking the oil, processing the oil by thermal depolymerization, adding the oil to a petroleum refining process, or a combination thereof.

30. The method of claim 29, wherein the biofuel is produced by transesterifying the oil.

31. The method of claim 29, wherein the biofuel is a biodiesel.

32. The method of claim 29, wherein the biofuel is produced by cracking the oil.

33. The method of claim 29, wherein the biofuel is a jet biofuel.

34. The method of claim 29, wherein the biofuel produced by processing the oil by thermal depolymerization.

35. The method of claim 29, wherein the biofuel is a renewable diesel.

36. The method of claim 29, wherein the biofuel produced by adding the oil to a petroleum relining process.

37. The method of claim 29, wherein the biofuel is a co-processed renewable diesel.
